(19) United States
(12) Patent Application Publication
(10) Pub. No.: US 2026/0209775 A1
(43) Pub. Date: Jul. 23, 2026

(54) ANTISENSE OLIGONUCLEOTIDES TARGETING EZH2 SPLICING AND THEIR USE FOR TREATING CANCER AND KIDNEY DISEASE

(71) Applicant: ASOcura Pharmaceuticals Suzhou Co., Ltd., Suzhou (CN)

(72) Inventor: Yimin HUA, Miami, FL (US)

(73) Assignee: ASOcura Pharmaceuticals Suzhou Co., Ltd., Suzhou (CN)

(21) Appl. No.: 19/631,930

(22) Filed: Mar. 27, 2026

Related U.S. Application Data
(63) Continuation of application No. PCT/CN2024/122452, filed on Sep. 29, 2024.
(60) Provisional application No. 63/587,012, filed on Sep. 29, 2023.

(51) Int. Cl. C12N 15/113; A61P 35/00; C12N 15/86

(52) U.S. Cl. CPC C12N 15/1137; A61P 35/00; C12N 15/86; C12Y 201/01043; C12N 2310/11; C12N 2310/314; C12N 2310/315; C12N 2310/321; C12N 2310/322; C12N 2310/3231; C12N 2310/3233; C12N 2310/3341; C12N 2320/33; C12N 2750/14143

(57) ABSTRACT

The present disclosure relates to the field of medicine. In particular, it relates to novel antisense oligonucleotides that promote exon 14 skipping in the EZH2 gene, and their use in the treatment of cancer and other diseases.

### CROSS REFERENCE TO RELATED APPLICATIONS

This application is a continuation of International Patent Application No. PCT/CN2024/122452, filed Sep. 29, 2024, which claims benefit of U.S. Provisional Patent Application No. 63/587,012, filed Sep. 29, 2023, both of which are incorporated herein by reference in their entirety.

### SEQUENCE LISTING

This application contains a Sequence Listing which is submitted electronically in XML format and is hereby incorporated by reference in its entirety. Said XML copy, created on Sep. 27, 2024, is named 2024-09-27_Sequence-Listing_144930-8003WO00.xml and is 60,434 bytes in size.

### FIELD OF THE DISCLOSURE

The present disclosure relates to novel antisense oligonucleotides (“ASOs”) that promotes exon 14 skipping of the EZH2 (enhancer of zeste homolog 2) gene, pharmaceutical compositions comprising one or more such ASOs, and their use in treating various conditions, e.g., without limitation, cancer, chronic kidney disease, acute kidney injury, neurodegeneration in ataxia-telangiectasia, retinal degeneration, and inflammation.

### BACKGROUND OF THE DISCLOSURE

Hepatocellular carcinoma (HCC) is the sixth most common malignancy and the third leading cause of cancer mortality worldwide. The HCC patient survival rate remains poor mainly owing to the limited treatment options. Chemotherapy, radiotherapy, surgical resection, and liver transplantation remain the mainstay of the HCC therapeutic strategies. However, the high recurrence and metastasis rates of these therapies make the survival and prognosis of patients, particularly with advanced HCC, unsatisfactory. Molecular-targeted therapies, represented by sorafenib, lenvatinib and some monoclonal antibodies also have issues such as limited efficacy, low response rates, and therapeutic resistance. Therefore, there is an urgent need to explore the molecular pathogenesis of HCC in order to discover improved therapeutic strategies. Xu L, Lin J, Deng W et al. EZH2 facilitates BMI1-dependent hepatocarcinogenesis through epigenetically silencing microRNA-200c. 2020. Oncogenesis 9, 101. doi: 10.1038/s41389-020-00284-w.

EZH2 is a polycomb group protein (PcG) that is encoded by the EZH2 gene. The protein is a central component of the polycomb repressive complex 2 (PRC2). It catalyzes the trimethylation of histone H3 at lysine 27 (H3K27), thereby repressing the transcription of target genes. An increase in EZH2 and H3K27me3 levels has been reported to be associated with aggressive tumor phenotypes and unfavorable prognosis. Kusakabe Y, Chiba T, Oshima M et al. EZH1/2 inhibition augments the anti-tumor effects of sorafenib in hepatocellular carcinoma. 2021 Nov. 1. Sci Rep. 11(1): 21396. doi: 10.1038/s41598-021-00889-0. Kim K H and Roberts C W. Targeting EZH2 in cancer. 2016. Nat. Med. 22(2), 128-134. doi: 10.1038/nm.4036. As the enzymatic catalytic subunit of PRC2, EZH2 is involved in global transcriptional repression, including tumor-suppressor genes. EZH2 is commonly overexpressed in cancer and shows activating mutations in subtypes of lymphoma. Eich M L, Athar M, Ferguson J E 3rd, Varambally S. EZH2-Targeted Therapies in Cancer: Hype or a Reality. 2020 Dec. 15. Cancer Res. 80(24): 5449-5458. doi: 10.1158/0008-5472.CAN-20-2147.

EZH2 is a substrate of ATM (A-T mutated) kinase and phosphorylation of EZH2 on Ser734 by ATM reduces its stability. It was found that EZH2-mediated increase of H3K27me3 is responsible for neurodegeneration in ataxia-telangiectasia. EZH2 and H3K27me3 also contribute to cell death in retinal degeneration. Li J, Hart R P, Mallimo E M, et al. EZH2-mediated H3K27 trimethylation mediates neurodegeneration in ataxia-telangiectasia. 2013 December. Nat Neurosci; 16(12): 1745-53. doi: 10.1038/nn.3564. Mbefo M, Berger A, Schouwey K, et al. Enhancer of Zeste Homoglog 2 (EZH2) contributes to rod photoreceptor death process in several forms of retinal degeneration and its activity can serve as a biomarker for therapy efficacy. 2021 Aug. 28. Int J Mol Sci. 22(17): 9331. doi: 10.3390/ijms22179331.

Upregulation of EZH2 and its substrate H3K27me3 is also involved in acute kidney injury. Inhibition of EZH2 attenuates renal fibrosis and prevents transition from acute kidney injury to chronic kidney disease through multiple signaling pathways downstream of EZH2/H3K27me3. Zhou X, Zang X, Ponnusam M., et al. Enhancer of zeste homolog 2 inhibition attenuates renal fibrosis by maintaining Smad7 and phosphatase and tensin homolog expression. 2016 July. J Am Soc Nephrol.; 27(7): 2092-108. doi: 10.1681/ASN.2015040457. Zhou X, Zang X, Guan Y, et al. Targeting enhancer of zeste homolog 2 protect against acute kidney injuey. 2018 Oct. 19. Cell Death Dis. 9(11): 1067. doi: 10.1038/s41419-018-1012-0.

In addition, EZH2 functions in regulation of the immune system. Inhibition of EZH2 ameliorates colitis and neuroinflammation. Nutt S L, Keenan C, and Chopin M. EZH2 function in immune cell development. 2020. Biol. Chem. doi: 10.1515/hsz-2019-0436. Zhou J, Huang S, Wang Z, et al. Targeting EZH2 histone methyltransferase activity alleviates experimental intestinal inflammation. 2019 Jun. 3. Nat Commun. 10(1): 2427. doi: 10.1038/s41467-019-10176-2. Luo Y, Fang Y, Kang R, et al. Inhibition of EZH2 (Enhancer of Zeste Homolog 2) Attenuates Neuroinflammation via H3k27me3/SOCS3/TRAF6/NF-κB (Trimethylation of Histone 3 Lysine 27/Suppressor of Cytokine Signaling 3/Tumor Necrosis Factor Receptor Family 6/Nuclear Factor-κB) in a Rat Model of Subarachnoid Hemorrhage. 2020 November. Stroke. 51(11): 3320-3331. doi: 10.1161/STROKEAHA.120.029951.

In eukaryotic genes containing coding (exons) and noncoding (intron) sequences, the noncoding introns are excised from the pre-mRNA transcript and the coding exons are spliced together to form mRNA. If an intron is left in the final mRNA transcript or an exon is left out, the mRNA reading frame may be disrupted during translation of the mRNA. This may result in a non-functional polypeptide sequence or a premature stop codon. The splicing process is further complicated by alternative splicing, where the same pre-mRNA sequence can be spliced into different exon combinations to form multiple mRNA sequences.

Splicing of pre-mRNA is an intricate process involving a multi-megadalton ribonucleoprotein complex called the spliceosome. The spliceosome recognizes specific sequences in pre-mRNA to precisely excise introns and ligate exons. The spliceosome catalyzes intron excision in two transesterification reactions using three conserved RNA sequences. These RNA sequences are the 5′ splice site, 3′ splice site, and the branch site. Will C L & Luhrmann R. Spliceosome structure and function. 2011 July. Cold Spring Harb. Perspect. Biol. 3(7), a003707. doi: 10.1101/cshperspect.a003707.

Splicing begins with the 2′ OH group of the branch site binding to the 5′ splice site via a nucleophilic attack, causing cleavage of the 5′ exon at the 5′ splice site and forming a lariat. Then the 3′ OH group of the 5′ exon attacks the 3′ exon at the 3′ splice site, ligating the 5′ and 3′ exons and cleaving the intron lariat. Will C L & Luhrmann R., Spliceosome structure and function. 2011 July. Cold Spring Harb. Perspect. Biol. 3(7), a003707. doi: 10.1101/cshperspect.a003707. Because the splicing process is entirely dependent on spliceosome recognition sites, 5′ and 3′ splice sites, and the branch site, a mutation in any one of these sites can disrupt the splicing process.

ASOs are polynucleotides designed to bind with specificity to a target nucleotide sequence, thereby affecting one or more aspects of gene expression, such as, transcription, splicing, stability, and/or translation. ASOs may be directed to either RNA or DNA. ASOs directed to RNA can bind to target mRNA sequences, effecting mRNA stability or translation at the ribosome.

ASOs that bind to target sequences in pre-mRNA transcripts can affect the splicing process. In some cases, ASOs may be used to prevent or reduce exon skipping during pre-mRNA splicing. As an example, the ASO drug nusinersen (Spinraza®) reduces exon 7 skipping during splicing of the SMN2 gene to treat spinal muscular atrophy. Hoy S M. Nusinersen: first global approval. 2017 March. Drugs. 77(4): 473-9. doi: 10.1007/s40265-017-0711-7.

ASOs may be utilized to correct the reading frame by inducing skipping of an exon during splicing. Removing an exon of the correct number of nucleotides results in a shorter mRNA transcript, but the reading frame may be corrected. Because dystrophin pre-mRNA consists of 79 exons, skipping one or several exons during splicing still results in a partly functional protein. The FDA approved an exon-skipping drug called Exondys 51 (eteplirsen) for treatment of DMD in 2016. Dowling J J. Eteplirsen therapy for Duchenne muscular dystrophy: skipping to the front of the line. 2016 December. Nature Review Neurology. 12(12), 675-676. doi: 10.1038/nrneurol. 2016.180.

There remains a need, however, for ASOs that successfully promotes exon 14 skipping of the EZH2 gene and for their use in treating related conditions such as cancer, chronic kidney disease, acute kidney injury, neurodegeneration in ataxia-telangiectasia, retinal degeneration, and inflammation.

### SUMMARY OF THE DISCLOSURE

The present disclosure relates to ASOs, methods of using such ASOs to promote exon 14 skipping of the EZH2 gene, pharmaceutical compositions that comprise one or more such ASOs, and methods of using such compositions to treat various conditions, e.g., without limitation, cancer, chronic kidney disease, acute kidney injury, neurodegeneration in ataxia-telangiectasia, retinal degeneration, and inflammation, etc.

In one aspect, the present disclosure provides ASOs of about 10 to about 30 nucleotides in length, and one of the lead ASOs is SEQ ID NO:1 (5′-CAAGGCTGCCGTGGATGATCACAG-3′). SEQ ID NO:1 is originally named as FY01631. In certain embodiments, the ASO comprises a sequence listed in Tables 1 or 2, but for Mismatch (SEQ ID NO: 19). In certain embodiments, the ASO is selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO: 11, SEQ ID NO:12, SEQ ID NO: 13, SEQ ID NO: 14, SEQ ID NO:15, SEQ ID NO: 16, SEQ ID NO: 17, SEQ ID NO: 18, SEQ ID NO:20, SEQ ID NO:21, SEQ ID NO:23, SEQ ID NO:24, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO: 58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, and SEQ ID NO:67 as shown in Tables 1 and 2.

In certain embodiments, the ASOs disclosed herein comprise at least a portion of non-natural backbone. In certain embodiments, the non-natural backbone comprises one or more modified sugar moieties. In certain embodiments, the one or more modified sugar moieties comprise one or more 2′-O-methoxyethyl ribose moieties (2′-MOE), 2′-O-methyl ribose moieties (2′-OMe), 2′-fluoro ribose moieties (2′-F), 4′-thioribosyl ribose moieties, locked nucleic acid oligos (LNA), constrained ethyl oligos (cEt), and/or morpholino rings. In certain embodiments, the non-natural backbone comprises one or more modified phosphates. In certain embodiments, the one or more modified phosphates comprise one or more phosphorothioates. In certain embodiments, the one or more modified phosphates comprise one or more phosphorodiamidates.

In certain embodiments, the ASOs disclosed herein comprise one or more modified nitrogenous bases. In certain embodiments, the modified nitrogenous bases may be 5-methylcytosine base.

In another aspect, the present disclosure provides a pharmaceutical composition comprising an ASO disclosed herein and a pharmaceutically acceptable carrier and/or excipient.

In another aspect, the present disclosure provides a method of promoting exon 14 skipping in the EZH2 gene, and/or downregulating H3K27me3 expression in vivo, comprising introducing a nucleic acid molecule into a cell, wherein the nucleic acid molecule is an ASO comprising all or a portion of SEQ ID NO:1, wherein the oligonucleotide hybridizes the CXC (cysteine-rich) region of the EZH2 gene, and wherein the oligonucleotide promotes exon 14 skipping of the EZH2 gene. In certain embodiments, the ASO comprises a sequence listed in Tables 1 or 2, but for Mismatch (SEQ ID NO: 19). In certain embodiments, the ASO is selected from the group consisting of SEQ ID NO: 1, SEQ ID NO:2, SEQ ID NO:3, SEQ ID NO:4, SEQ ID NO:5, SEQ ID NO:6 SEQ ID NO: 7, SEQ ID NO:8 SEQ ID NO:9, SEQ ID NO: 10, SEQ ID NO:11, SEQ ID NO:12, SEQ ID NO: 13, SEQ ID NO: 14, SEQ ID NO: 15, SEQ ID NO:16, SEQ ID NO: 17, SEQ ID NO: 18, SEQ ID NO: 20, SEQ ID NO:21, SEQ ID NO:23, SEQ ID NO:24, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO: 60, SEQ ID NO:61, SEQ ID NO: 62, SEQ ID NO: 63, SEQ ID NO:64, SEQ ID NO:65, SEQ ID NO:66, and SEQ ID NO:67. In certain embodiments, the cell may be an animal cell, e.g., without limitation, a human cell. In certain embodiments, the nucleic acid molecule is introduced into the cell by way of an expression vector. Examples of the expression vector includes, without limitation, a pCI-neo expression vector or an AAV (adeno-associated virus) vector.

In another aspect, the present disclosure provides a method of treating a condition that is related to the exon 14 skipping in the EZH2 gene and/or the downregulation of H3K27me3 expression in a subject comprising administering the subject with a therapeutically effective amount of an ASO comprising all or a portion of SEQ ID NO:1. In certain embodiments, the ASO comprises a sequence listed in Tables 1 or 2, but for Mismatch (SEQ ID NO: 19). In certain embodiments, the ASO is selected from the group consisting of SEQ ID NO:1, SEQ ID NO:2, SEQ ID NO:3, SEQ ID NO:4, SEQ ID NO:5, SEQ ID NO:6 SEQ ID NO:7, SEQ ID NO:8 SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO:16, SEQ ID NO: 17, SEQ ID NO: 18, SEQ ID NO:20, SEQ ID NO:21, SEQ ID NO:23, SEQ ID NO:24, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO: 43, SEQ ID NO:44, SEQ ID NO:45, SEQ ID NO:46, SEQ ID NO:47, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO: 62, SEQ ID NO: 63, SEQ ID NO:64, SEQ ID NO:65, SEQ ID NO:66, and SEQ ID NO:67. In certain embodiments, the ASO is administered via parenteral administration. Examples of such conditions include, without limitation, cancer, (e.g., without limitation, liver cancer, lung cancer, breast cancer, ovarian cancer, endometrial cancer, melanoma, NK/T-cell lymphoma, non-Hodgkin's lymphoma, prostate cancer, bladder cancer, gastric cancer, renal cancer, brain cancer, colorectal cancer, and pancreatic cancer), chronic kidney disease, acute kidney injury, neurodegeneration in ataxia-telangiectasia, retinal degeneration, and inflammation.

### BRIEF DESCRIPTION OF THE FIGURES

FIG. 1A is a schematic representation of the EZH2 gene and EZH2 protein domain structures. FIG. 1B is a schematic representation of the effect by an example of an optimal ASO disclosed herein (FY01631 (SEQ ID No:1)) on exon 14 skipping. FIG. 1C shows positions for the forward (F) and reverse (R) primers. FIG. 1D shows the effects of ASO FY01631 (50 nM) and a mismatch ASO (Mismatch, 50 nM) on EZH2 exon 14 splicing; buffer (without adding ASO) was used as negative control. ASO FY01631 and Mismatch were respectively transfected into a MHCC97H human hepatocellular carcinoma cell line using Exfect Transfection Reagent (Vazyme Biotech, Nanjing, China) and splicing was analyzed by semiquantitative fluorescent RT-PCR and native PAGE. Mismatch (SEQ ID NO:19) is a mismatch version of FY01631, containing 8 mismatched nucleotides compared to FY01631 (see Table 1). FL, full-length transcript; A14, exon 14-skipped transcript; % excl, percentage of exclusion. All schematics, including exons, introns, structural domains, etc., are not drawn to scale.

FIGS. 2A-2G illustrate the screening of ASOs that promoted exon 14 skipping of the EZH2 gene in cultured human MHCC97H cells. FIG. 2A shows alignments of the ASOs tested in the initial ASO screening experiments described herein. FIG. 2B shows a representative PAGE demonstrating the effect of nine ASOs used for the first-round screening with buffer and an unrelated scrambled ASO (5′-TTGTATTCTATGTTT-3′) (Con) as controls. FIG. 2C shows the quantitation of the ASO initial screening in FIG. 2B and two more repeated independent experiments of same. FIG. 2D shows microwalk screening conducted for FY01604 (SEQ ID No:7).

FIG. 2E shows the quantitation of the microwalk screening data in FIG. 2D. FIG. 2F shows microwalk screening conducted for FY01606 (SEQ ID No:14). FIG. 2G shows the quantitation of the microwalk screening data in FIG. 2F. Information of ASOs used in FIGS. 2A-2G is shown in Table 1. FL: full length; Δ14: transcript with exon 14 skipping. For all statistics, n=3, *P<0.05, **P<0.01, ***P<0.001 versus buffer.

FIGS. 3A-3B illustrate length-optimization screening of ASOs that promote EZH2 exon 14 skipping in cultured human MHCC97H cells. FIG. 3A shows a representative PAGE. FIG. 3B shows the quantitation data by % excl. 35 additional ASOs ranging from 21- to 28-nt long targeting the region from position 31 to 58 in EZH2 exon 14 were tested. FY01611 and FY01631 was used as positive controls; the scrambled ASO (Con) as shown in FIGS. 2B-2G and buffer were used as negative controls. Multiple potent ASOs were identified. For all statistics, n=3, ***P<0.001 versus buffer.

FIGS. 4A-4C illustrate the growth inhibition effect of FY01631 in HCC cells in vitro. FIG. 4A shows images of cell colony formation of HCC cells (MHCC97H and HCCLM3 cells) treated with or without FY01631. Cells were first transfected with FY01631 (50 nM), Mismatch (50 nM), or buffer and incubated for 48 hours. And then 5000 cells of each group were seeded into 6-well plates and incubated for 10 days, followed by crystal violet staining and cell counting. FIG. 4B shows the quantitation of data shown in FIG. 4A. FIG. 4C shows live cells quantitated by the tetrazolium assay (Cell Counting Kit-8) on HCC cells (MHCC97H and HCCLM3 cells in 96-well plates) transfected with FY01631 (50 nM), Mismatch (50 nM) or buffer. Cells were incubated for 0, 24, 48 or 72 hours (h) post transfection. Absorbance was measured at 450 nm by a microplate reader. For all groups, n=3; ***P<0.001 versus buffer.

FIGS. 5A-5D illustrate apoptosis of tumor cells markedly promoted by FY01631. FIG. 5A shows hepatocellular carcinoma cell lines MHCC97H transfected with FY01631 (50 nM), Mismatch (50 nM), and buffer, respectively. FIG. 5B shows hepatocellular carcinoma cell lines HCCLM3 transfected with FY01631 (50 nM), Mismatch (50 nM), and buffer, respectively. Cells were incubated for 48 hours, and then stained with propidium iodine (PI) and FITC-Annexin V, followed by flow cytometry analysis. FIG. 5C shows the quantitation of apoptotic cells shown in FIG. 5A. FIG. 5D shows the quantitation of apoptotic cells shown in FIG. 5B. For all groups, n=3; *P<0.05, ***P<0.001 versus buffer.

FIGS. 6A-6D show that FY01631 boosted cell cycle G2/M arrest in tumor cells. FIG. 6A shows cell cycle analysis by flow cytometry, using propidium iodine to stain the hepatocellular carcinoma cell lines MHCC97H 48 hours post transfection with FY01631 (50 nM), Mismatch (50 nM), and buffer alone, respectively. FIG. 6B shows cell cycle analysis by flow cytometry, using propidium iodine to stain the hepatocellular carcinoma cell lines HCCLM3 48 hours post transfection with FY01631 (50 nM), Mismatch (50 nM), and buffer alone, respectively. FIG. 6C shows the quantitation of FIG. 6A. FIG. 6D shows the quantitation of FIG. 6B. For all groups, n=3; ***P<0.001 versus buffer (G2/M phase).

FIGS. 7A-7E show that FY01631 promoted mouse Ezh2 exon 14 skipping and downregulated H3K27me3 expression in vivo. FIG. 7A shows the dosing schedule for six-week-old male BALB/c mice, which were injected subcutaneously with FY01631 at 100 μg/g per day for eight consecutive days, and saline was used as a negative control. FIG. 7B shows RT-PCR analysis of tissue samples (liver, lung, kidney, and brain) collected after euthanasia performed 3 days after the last injection. FIG. 7C shows Western blotting analysis of EZH2 and its downstream protein H3K27me3 in the liver, lung, kidney, and brain samples; GAPDH was used as loading control. FIG. 7D shows the quantitation of splicing data from three independent experiments, as shown in FIG. 7B. FIG. 7E shows the quantitation of data from three mice as shown in FIG. 7C. All protein levels were first normalized to GAPDH and then normalized to saline control with the protein levels in saline group being set as 1. For all groups, n=3; ***P<0.001 versus saline.

FIGS. 8A-8G show that FY01631 inhibited liver tumor growth and extended survival in a xenograft mouse model, and the effects of FY01631 on the body weight, liver weight, and ratio of liver weight to body weight. FIG. 8A shows the FY01631 dosing schedule after luciferase-labeled MHCC-97H cells being injected into liver of 6-week-old male mice (n=16 for each group) that created the xenograft mouse model. Mismatch and saline were used as controls. In each group, 12 mice were pre-set to evaluate survival after final imaging and 4 were pre-set to analyze tumor weight and histological changes (also refer to FIGS. 9A-9D). FIG. 8B shows the whole-body live imaging of representative mice performed on days 9, 13, 17, and 20, respectively. FIG. 8C shows the total fluorescence intensity statistics (n=16) as shown in FIG. 8B. The vertical coordinate (ATotal Flux [p/s]) refers to changes in Total Flux [p/s] in mice at days 13, 17, and 20 compared to Day 9. Total Flux refers to the radiance (photons/sec) in each pixel summed or integrated over the ROI area (cm²)×4π. FIG. 8D shows the survival curve (n=12) analyzed by the log-rank test. FY01631 treated mice had median survival of 52 days, significantly longer than both saline-treated (44 days, P<0.0001) and Mismatch-treated (46 days, P<0.0001)) mice. FIG. 8E shows the body weight recorded for each mouse at the time of sacrifice (n=4). FIG. 8F shows the liver weight recorded for each mouse at the time of sacrifice (n=4). FIG. 8G shows the ratio of liver weight to body weight recorded for each mouse at the time of sacrifice (n=4). *P<0.05, **P<0.01, **P<0.01; ns: no significance.

FIGS. 9A-9D show the effect of FY01631 on tumor morphology and levels of EZH2, H3K27me3 and Ki67 in xenograft mice created in FIG. 8A (n=4). FIG. 9A shows H&E staining images of liver tumor sections. FIG. 9B shows immunohistochemistry of liver tumor sections for EZH2 and H3K27me3 using a specific monoclonal antibody for each protein. FIG. 9C shows immunohistochemistry of liver tumor sections for Ki67. FIG. 9D shows the quantitation of the data (n=4) in FIG. 9C. Tissue samples were collected at day 20 as indicated in FIG. 8A. Nuclei were counterstained by hematoxylin. **P<0.01, ***P<0.001.

FIGS. 10A-10G show that treatment of FY01631 improved renal function in mice with chronic kidney disease (CKD) induced by adenine (Ade) treatment. FIG. 10A shows the schedules of Ade feeding, FY01631 treatment, and sampling time point. After one week of environment habituation, 8-week-old C57BL/6J mice were placed on a diet containing 0.2% Ade for 28 days to induce CKD, followed by subcutaneous injection of FY01631 at 200 mg/kg on day 35, day 37, day 39, and day 41. Four mice of each group were sacrificed for tissue sample analysis and remaining mice were monitored for survival. Mice fed with normal chow without adding Ade (w/o Ade) were used as normal control mice. FIG. 10B shows that Ezh2 exon 14 skipping was enhanced in mouse kidney samples after treatment of FY01631 using RT-PCR analysis. FIG. 10C shows H&E staining (left) and tubular injury scores (right) of kidney tissues (scale bar=100 μm). Ade treatment caused tissue damages including renal tubular dilation, renal tubular epithelial cell necrosis and detachment as showed in saline and Mismatch groups. FY01631 treatment alleviated the symptoms. FIG. 10D shows improvement of renal function after FY01631 treatment by measuring serum creatine (sCr) and blood urea nitrogen (BUN) levels. FIG. 10E shows mRNA expression reduction of two kidney tubular injury markers Kim-1 and NGAL after FY01631 treatment using quantitative RT-PCR (qRT-PCR). FIG. 10F shows mRNA expression changes of proinflammatory cytokines IL-6, IL-1β, TNF-α, and MCP-1 in kidney samples after FY01631 treatment using qRT-PCR. For FIGS. 10E-10F, GAPDH was used as an internal control and the mRNA ratio in the w/o Ade group was set as 1. FIG. 10G shows that FY01631 treatment extended the median survival of mice (n=7 for each group) with CKD from 68 days (saline treatment) or 69 days (Mismatch treatment) to 92 days. *P<0.05, **P<0.01, ***P<0.001; ns: no significance

FIGS. 11A-11E show that treatment of FY01631 improved renal function in mice with acute kidney injury (AKI) induced by cisplatin treatment. FIG. 11A shows the schedules of cisplatin injection, FY01631 treatment, and sampling time point. After three days of environmental habituation, 8-week-old BALB/c mice were intraperitoneally injected with cisplatin at 20 mg/kg on day 3; cisplatin-injected mice were further administered subcutaneously with saline (cisplatin) or FY01631 at 150 mg/kg (cisplatin+FY01631) from day 3 for three consecutive days. Mice received neither cisplatin nor FY01631 were used as normal controls. On day 10, mice were euthanized, and kidney tissues and serum were collected for further analysis. FIG. 11B shows that Ezh2 exon 14 skipping in kidney was increased after treatment of FY01631. FIG. 11C shows reduced renal tubular damage after FY01631 treatment (scale bar=40 μm). FIG. 11D shows mRNA expression changes of Kim-1, NAGL, and IL-6 after FY01631 treatment (n=3). GAPDH was used as an internal control and the mRNA ratio in the normal control mice was set as 1. FIG. 11E show reduction of serum creatinine (sCr) and blood urea nitrogen (BUN) to normal levels after FY01631 treatment (n=4). ***P<0.001.

### DETAILED DESCRIPTION OF THE DISCLOSURE

### Definitions

The term “oligonucleotide” is used herein to refer to a nucleotide sequence comprising at least ten DNA or RNA nucleotides.

The term “antisense oligonucleotide,” abbreviated as “ASO,” is used herein to refer to a nucleotide sequence comprising an antisense sequence that is sufficiently complementary to a target nucleotide sequence in order to form a stable double stranded hybrid with the target nucleotide sequence. In some embodiments, the target nucleotide sequence is an RNA nucleotide sequence.

The term “nucleobase” is used herein to refer to a nitrogenous base that is a component of a nucleoside. Example nucleobases include, but are not limited to, adenine, guanine, thymine, cytosine, and uracil.

The term “nucleoside” is used herein to refer to a nucleobase covalently linked to a sugar. Examples of naturally occurring and non-natural nucleosides are described below.

The term “nucleotide” is used herein to refer to a nucleoside covalently linked to a phosphate group. Examples of naturally occurring and non-natural nucleotides are described below.

The term “non-natural” is used herein to refer to one or more nucleotide subunits having at least one modification selected from (i) a modified internucleotide linkage, e.g., an internucleotide linkage other than the standard phosphodiester linkage found in naturally-occurring oligonucleotides, (ii) modified sugar moieties, e.g., moieties other than ribose or deoxyribose moieties found in naturally occurring oligonucleotides, (iii) modified nitrogenous bases, e.g., bases other than those found in naturally occurring oligonucleotides, or (iv) a combination of the foregoing.

The term “morpholino” is used herein to refer to a nucleobase that contains a morpholinyl ring instead of a ribose.

The term “complementary” is used herein to describe when the corresponding positions of at least two nucleotide sequences are occupied by nucleotides which can hydrogen bond with each other.

The term “hybridize” is used herein to describe the binding of two complementary nucleotide sequences, forming one double stranded molecule. When a sufficient number of corresponding nucleotides in two sequences can hydrogen bond with each other in order, i.e., they are sufficiently complementary, they may form a stable hybrid. It is understood in the art that 100% complementarity is not necessary for an ASO to hybridize with a target sequence.

The term “sufficient complementarity” is used herein to indicate a level of complementarity sufficient to permit an ASO to specifically bind to its target sequence and form a stable hybrid. In one embodiment, the complementarity of the ASO and the target sequence is at least 99%, or 98%, or 97%, or 96%, or 95%, or 94%, or 93%, or 92%, or 91%, or 90%, or 89%, or 88%, or 87%, or 86%, or 85%, or 84%, or 83%, or 82%, or 81%, or 80%, or 79%, or 78%, or 77%, or 76%, or 75%, or 74%, or 73%, or 72%, or 71%, or 70%.

The terms “target region” and “target sequence” are used interchangeably herein to designate a nucleotide sequence to which an ASO will hybridize under physiological conditions. It is not necessary for the ASO and the target region to be 100% complementary, so long as there is sufficient complementarity for the ASO to hybridize to the target sequence and form a stable hybrid. The ASO may hybridize to all or a portion of the target sequence.

The terms “treat,” “treating,” or “treatment” are used herein to refer to ameliorating a disease or disorder (i.e., slowing or arresting or reducing the development of the disease or at least one of the clinical symptoms thereof). The terms also refer to alleviating or ameliorating at least one physical parameter including those which may not be discernible by the patient. The terms also refer to modulating the disease or disorder, either physically (e.g., through stabilization of a discernible symptom), physiologically, (e.g., through stabilization of a physical parameter), or both. The terms also refer to preventing or delaying the onset or development or progression of the disease or disorder.

The term “therapeutically effective amount” is used herein to refer to the amount of a therapeutic agent or composition effective in prevention or treatment of a disorder or disease. In one embodiment, this includes an amount of a therapeutic agent or composition effective in the prevention or treatment of cancer, chronic kidney disease, acute kidney injury, neurodegeneration in ataxia-telangiectasia, retinal degeneration, and inflammation.

The term “pharmaceutically acceptable” is used herein to refer to a molecular entity or composition that is pharmaceutically useful and not biologically or otherwise undesirable.

The term “carrier” is used herein to refer to a diluent, adjuvant, excipient, or vehicle with which the compound is administered.

The term “excipient” as used herein refers to any ingredient in a pharmaceutical composition other than the active ingredient.

The term “about” is used herein to refer to a quantity, level, value, number, frequency, percentage, dimension, size, amount, weight or length that varies by acceptable levels in the art. In some embodiments, such variation may be as much as 30, 25, 20, 15, 10, 9, 8, 7, 6, 5, 4, 3, 2 or 1% to a reference quantity, level, value, number, frequency, percentage, dimension, size, amount, weight or length. When the term “about” is used in conjunction with a numerical range, it modifies that range by extending the boundaries above and below the numerical values set forth.

Unless otherwise defined, all other scientific and technical terms have the same meaning as commonly understood to one of ordinary skill in the art. Such scientific and technical terms are explained in the literature, for example: Sambrook J, Fritsch E F, and Maniatis T, 1989, Molecular Cloning: A Laboratory Manual, Second Edition, Books 1-3, Cold Spring Harbor Laboratory Press; Martin, 1990, Remington's Pharmaceutical Sciences, 18th Edition, Mack Publishing Co; Glover D M. 1985. DNA Cloning: A Practical Approach. Volumes I and II, MRL Press, Ltd.; and Ausubel F, Brent R, Kingston R E, Moore D D, Seidman J G, Smith J A, Struhl K. 2002. Current Protocols in Molecular Biology. Greene Publishing Associates/Wiley Intersciences.

### Antisense Oligonucleotides (ASOs)

In one aspect, disclosed herein are ASOs directed to a target sequence in the EZH2 pre-mRNA. In certain embodiments, the ASOs target all or a portion of a nucleotide target sequence of exon 14 in the EZH2 pre-mRNA (Table 1). Two target regions were identified in the exon: one is from position 31 to position 58, and the other from position 67 to position 93. The two regions, when intact or not occupied by ASOs, keep exon 14 in the final transcript, suggesting the presence of splicing enhancer motifs in the two regions.

In another embodiment, ASOs that are directed to the target sequence form a sufficiently stable hybrid with the target sequence and are about 15 to about 30 nucleotides in length. In certain examples, these ASOs may be sufficiently complimentary to all or a portion of the target sequence.

In certain embodiments, the ASO comprises a sequence listed in Tables 1 or 2, but for Mismatch (SEQ ID NO: 19).

In certain embodiments, the ASO comprises a sequence selected from the group consisting of SEQ ID NO: 1, SEQ ID NO:2, SEQ ID NO:3, SEQ ID NO:4, SEQ ID NO:5, SEQ ID NO:6 SEQ ID NO: 7, SEQ ID NO:8 SEQ ID NO:9, SEQ ID NO: 10, SEQ ID NO: 11, SEQ ID NO: 12, SEQ ID NO: 13, SEQ ID NO:14, SEQ ID NO: 15, SEQ ID NO: 16, SEQ ID NO: 17, SEQ ID NO: 18, SEQ ID NO: 20, SEQ ID NO:21, SEQ ID NO:23, SEQ ID NO:24, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO: 62, SEQ ID NO: 63, SEQ ID NO:64, SEQ ID NO:65, SEQ ID NO:66, and SEQ ID NO:67 (Tables 1 and 2).

In some embodiments, at least some nucleobases of the ASOs will replace thymine with uracil, or uracil with thymine. In some embodiments, at least some nucleosides of the ASOs will replace deoxyribose with ribose, or ribose with deoxyribose.

### Nucleotide Modifications

In another embodiment, the disclosed ASOs comprise one or more nucleotides that are chemically modified in one or more ways known to those of skill in the art. Nucleotide modifications include, for example, modified nitrogenous bases, sugar moieties, and phosphates. Such modifications are preferable at least in their ability to resist nuclease degradation.

Specific examples of chemically modified ASOs useful in this disclosure include ASOs containing modified phosphate backbones or non-natural inter-nucleoside linkages. ASOs having modified backbones include those that retain a phosphorus atom in the backbone and those that do not have a phosphorus atom in the backbone.

In other embodiments, both the sugar and the inter-nucleoside linkage, i.e., the backbone, of the nucleotide units in the ASO are replaced with novel groups. The base units are maintained for hybridization with an appropriate nucleic acid target compound. For example, the ASO may be a peptide nucleic acid (PNA). In PNA compounds, the sugar-backbone of an oligonucleotide is replaced with an amide containing backbone, for example, an aminoethylglycine backbone. The nucleo-bases are retained and are bound directly or indirectly to aza nitrogen atoms of the amide portion of the backbone.

Modified ASOs may also contain one or more substituted sugar moieties, for example, one or more sugar moieties that are mono- or disubstituted at the 2′, 3′ and/or 5′ position such as a —OH; —F; substituted or unsubstituted, linear or branched lower (C1-C10) alkyl, alkenyl, alkynyl, alkaryl, allyl, or aralkyl, that may be interrupted by one or more heteroatoms; O—, S—, or N-alkyl; O—, S—, or N-alkenyl; O—, S— or N-alkynyl; O—, S—, or N-allyl; O-alkyl-O-alkyl, -methoxy, -aminopropoxy; methoxyethoxy; dimethylaminooxyethoxy; and -dimethylaminoethoxyethoxy. The sugar moiety can be a pyranose or derivative thereof, or a deoxypyranose or derivative thereof, or a ribose or derivative thereof, or a deoxyribose or derivative thereof. In one embodiment, the substituted sugar moiety is a 2′-O-methoxyethyl ribose moiety (2′-MOE), 2′-O-methyl ribose moiety (2′-OMe), 2′-fluoro ribose moiety (2′-F), 4′-thioribosyl ribose moiety, locked nucleic acid oligo (LNA), or constrained ethyl oligo (cEt).

Modified ASOs may also contain one or more nucleobase (often referred to in the art simply as “base”) modifications or substitutions, for example, 5-substituted pyrimidines, 6-azapyrimidines and N-2, N-6 and O-6 substituted purines, including 2-aminopropyladenine, 5-propynyluracil and 5-propynylcytosine. Certain nucleobases are particularly useful for increasing the binding affinity of the oligomeric compounds of the disclosure. 5-methylcytosine substitutions have been shown to increase nucleic acid duplex stability by 0.6-1.2° C.

Another modification of the ASOs of the disclosure involves chemically linking to the ASO one or more moieties or conjugates that enhance the activity, cellular distribution or cellular uptake of the ASO. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

Another modification to the ASOs include morpholino-based ASOs. Morpholino-based oligomers refer to an oligomer comprising morpholino subunits supporting a nucleobase and, instead of a ribose, contains a morpholinyl ring. Exemplary internucleotide linkages include, for example, phosphoramidate or phosphorodiamidate internucleotide linkages joining the morpholinyl ring nitrogen of one morpholino subunit to the 4′ exocyclic carbon of an adjacent morpholino subunit. Each morpholino subunit comprises a purine or pyrimidine nucleobase effective to bind, by base-specific hydrogen bonding, to a base in an oligonucleotide.

Morpholino-based ASOs (including modified ASOs) are detailed, for example, in U.S. Pat. Nos. 5,698,685; 5,217,866; 5,142,047; 5,034,506; 5,166,315; 5,185,444; 5,521,063; 5,506,337, which are hereby incorporated by reference in their entirety.

Within the ASO structure, the phosphate groups are commonly referred to as forming the “internucleotide linkages” of the ASO. The naturally occurring internucleotide linkage of RNA and DNA is a 3′ to 5′ phosphodiester linkage. A “phosphoramidate” group comprises phosphorus having three attached oxygen atoms and one attached nitrogen atom, while a “phosphorodiamidate” group comprises phosphorus having two attached oxygen atoms and two attached nitrogen atoms. A “phosphorotriamidate” group (or a phosphoric acid triamide group) comprises phosphorus having one attached oxygen atom and three attached nitrogen atoms. In the uncharged or the cationic internucleotide linkages of the morpholino-based ASOs described herein, one nitrogen is always pendant to the linkage chain. The second nitrogen, in a phosphorodiamidate linkage, is typically the ring nitrogen in a morpholino ring structure.

It is not necessary for all positions in a given ASO to be uniformly modified, and in fact more than one of the aforementioned modifications may be incorporated in a single nucleoside within an ASO. ASOs may contain at least one region wherein the ASO is modified to confer upon them increased resistance to nuclease degradation, increased cellular uptake, and/or an additional region for increased binding affinity for the target nucleic acid.

### Manufacturing Antisense Oligonucleotides

The antisense molecules used in accordance with this disclosure may be made through well-known techniques of solid phase synthesis. Equipment for such synthesis is available from several sources including, for example, Applied Biosystems (Foster City, Calif.). One method for synthesizing oligonucleotides on a modified solid support is described in U.S. Pat. No. 4,458,066.

Any other means for such synthesis known in the art may additionally or alternatively be employed. It is well known to use similar techniques to prepare oligonucleotides such as the phosphorothioates and alkylated derivatives. In one such automated embodiment, diethyl-phosphoramidites are used as starting materials and may be synthesized as described by Beaucage S L, Caruthers M H. Deoxynucleoside phosphoramidites—A new class of key intermediates for deoxypolynucleotide synthesis. 1981. Tetrahedron Letters, 22(20): 1859-1862. doi: 10.1016/S0040-4039(01)90461-7.

The ASOs of the disclosure are synthesized in vitro and do not include antisense compositions of biological origin. The ASOs of the disclosure may also be mixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption.

### Pharmaceutical Composition of ASOs

In another aspect, the present disclosure provides a pharmaceutical composition comprising an ASO disclosed herein and a pharmaceutically acceptable carrier and/or excipient, as disclosed herein.

### Methods of Promoting Exon 14 Skipping of the EZH2 Gene

In another aspect, the present disclosure provides a method of promoting exon 14 skipping in the EZH2 gene, and/or downregulating H3K27me3 expression in vivo, comprising introducing a nucleic acid molecule or a pharmaceutical composition thereof into a cell.

In certain embodiments, the nucleic acid molecule is an ASO that hybridizes to a portion of the CXC (cysteine-rich)-coding region of the EZH2 gene and promotes exon 14 skipping of the EZH2 gene. In certain embodiments, the ASO comprises all or a portion of SEQ ID NO:1. In certain embodiments, the ASO comprises a sequence listed in Tables 1 or 2, but for Mismatch (SEQ ID NO: 19). In another embodiment, the ASO comprises one or more nucleotide sequences selected from the group consisting of SEQ ID NO:1, SEQ ID NO:2, SEQ ID NO:3, SEQ ID NO:4, SEQ ID NO:5, SEQ ID NO:6 SEQ ID NO:7, SEQ ID NO:8 SEQ ID NO:9, SEQ ID NO: 10, SEQ ID NO: 11, SEQ ID NO:12, SEQ ID NO: 13, SEQ ID NO: 14, SEQ ID NO:15, SEQ ID NO:16, SEQ ID NO: 17, SEQ ID NO: 18, SEQ ID NO:20, SEQ ID NO:21, SEQ ID NO:23, SEQ ID NO:24, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, and SEQ ID NO:67.

In one embodiment, the ASO is administered by itself, as a so-called “naked” ASO. The naked ASO is synthesized in vitro.

In another embodiment, the ASO is administered in the form of an expression vector, wherein the expression vector encodes an RNA transcript comprising the sequence of said ASO according to the disclosure. When placed under conditions conducive to expression of the encoded ASO, the expression vector can express the encoded ASO, which can hybridize to at least a portion of the CXC (cysteine-rich)-coding region of the EZH2 gene to promote exon 14 skipping. The expression vector can be a viral or non-viral vector. In certain embodiments, the expression vector may be a plasmid-based expression vector comprising an expression cassette or a transcription cassette that drives expression or transcription of an ASO for redirecting splicing according to the disclosure. In certain embodiments, the expression vector is a pCI-neo expression vector.

A cell can be provided with an ASO for redirecting splicing according to the disclosure by plasmid-derived ASO expression or viral expression provided by cytolomegalovirus-, adenovirus- or adeno-associated virus-based vectors. Expression may be driven by an RNA polymerase II promoter (Pol II) such as a U7 RNA promoter or an RNA polymerase III (Pol III) promoter, such as a U6 RNA promoter. In one embodiment, the delivery vehicle is a vector such as a pCI-neo vector and the like. Also, plasmids and artificial chromosomes are usable for targeted homologous recombination and integration in the human genome of cells may be suitably applied for delivery of an ASO for redirecting splicing according to the disclosure.

Methods of introducing “naked” ASOs or expression vectors encoding ASOs into a cell are well known in the art. An ASO or expression vector encoding an ASO can be introduced by transfection using known transfection agents. In one embodiment, the use of an excipient or transfection agent aids in delivery of the ASO or expression vector encoding the ASO as defined herein to a cell and/or into a cell. In another embodiment, excipients or transfection agents are capable of forming complexes, nanoparticles, micelles, vesicles and/or liposomes that deliver each ASO or expression vector encoding each ASO as defined herein, complexed or trapped in a vesicle or liposome through a cell membrane. Many of these excipients are known in the art. Suitable excipients or transfection agents include, but are not limited to, LipofectAMINE™ 2000 (Invitrogen), polyethylenimine (PEI; ExGen500 (MBI Fermentas)), or derivatives thereof, or similar cationic polymers, including polypropyleneimine or polyethylenimine copolymers (PECs) and derivatives, synthetic amphiphils (SAINT-18), Lipofectin™, DOTAP and/or viral capsid proteins that are capable of self-assembly into particles that can deliver each ASO or expression vector encoding each ASO as defined herein to a cell. Such excipients have been shown to efficiently deliver an oligonucleotide such as ASOs to a wide variety of cultured cells. Their high transfection potential is combined with an excepted low to moderate toxicity in terms of overall cell survival. The ease of structural modification can be used to allow further modifications and the analysis of their further (in vivo) nucleic acid transfer characteristics and toxicity.

Methods of Treating Conditions Related to the Exon 14 Skipping in the EZH2 Gene and/or the Downregulation of H3K27Me3 Expression

In another aspect, the present disclosure provides a method of treating a condition that is related to the exon 14 skipping in the EZH2 gene and/or the downregulation of H3K27me3 expression in a subject comprising administering the subject with a therapeutically effective amount of an ASO or a pharmaceutical composition thereof, wherein the ASO hybridizes to a portion of the CXC (cysteine-rich)-coding region of the EZH2 gene and promotes exon 14 skipping of the EZH2 gene. In certain embodiments, the ASO comprises all or a portion of SEQ ID NO:1. In certain embodiments, the ASO comprises a sequence listed in Tables 1 or 2, but for Mismatch (SEQ ID NO: 19). In another embodiment, the ASO comprises one or more nucleotide sequences selected from the group consisting of SEQ ID NO:1, SEQ ID NO:2, SEQ ID NO:3, SEQ ID NO:4, SEQ ID NO:5, SEQ ID NO:6 SEQ ID NO:7, SEQ ID NO:8 SEQ ID NO:9, SEQ ID NO: 10, SEQ ID NO: 11, SEQ ID NO:12, SEQ ID NO:13, SEQ ID NO: 14, SEQ ID NO:15, SEQ ID NO:16, SEQ ID NO: 17, SEQ ID NO: 18, SEQ ID NO:20, SEQ ID NO:21, SEQ ID NO:23, SEQ ID NO:24, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO: 42, SEQ ID NO:43, SEQ ID NO:44, SEQ ID NO: 45, SEQ ID NO:46, SEQ ID NO:47, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO: 60, SEQ ID NO: 61, SEQ ID NO:62, SEQ ID NO:63, SEQ ID NO:64, SEQ ID NO:65, SEQ ID NO:66, and SEQ ID NO:67.

In one embodiment, the ASO is administered by itself, as a so-called “naked” ASO as described herein.

In another embodiment, the ASO is administered in the form of an expression vector as described herein, wherein the expression vector encodes an RNA transcript comprising the sequence of said ASO according to the disclosure.

Examples of the conditions include, without limitation, cancer, chronic kidney disease, acute kidney injury, neurodegeneration in ataxia-telangiectasia, retinal degeneration, and inflammation, etc.

Examples of cancers include, without limitation, liver cancer, lung cancer, breast cancer, ovarian cancer, endometrial cancer, melanoma, NK/T-cell lymphoma, non-Hodgkin's lymphoma, prostate cancer, bladder cancer, gastric cancer, renal cancer, brain cancer, colorectal cancer, and pancreatic cancer.

The amount of ASO administered in a pharmaceutical composition may be dependent on the subject being treated, the subject's weight, the manner of administration and the judgment of the prescribing physician. For example, a dosing schedule may involve the daily or semi-daily administration of the pharmaceutical composition at a perceived dosage of about 1 μg to about 1000 mg. In another embodiment, intermittent administration, such as on a monthly or yearly basis, of a dose of the pharmaceutical composition may be employed. In accordance with standard dosing regimens, physicians will readily determine optimum dosages and will be able to readily modify administration to achieve such dosages.

A therapeutically effective amount of a compound or composition disclosed herein can be measured by the therapeutic effectiveness of the compound. The dosages, however, may be varied depending upon the requirements of the patient, the severity of the condition being treated, and the compound being used. In one embodiment, the therapeutically effective amount of a disclosed compound is sufficient to establish a maximal plasma concentration. Preliminary doses as, for example, determined according to animal tests, and the scaling of dosages for human administration is performed according to art-accepted practices.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

Data obtained from the cell culture assays or animal studies can be used in formulating a range of dosage for use in humans. Therapeutically effective dosages achieved in one animal model may be converted for use in another animal, including humans, using conversion factors known in the art (see, e.g., Freireich E J, Gehan E A, Rall D P, Schmidt L H, Skipper H E. Quantitative comparison of toxicity of anticancer agents in mouse, rat, hamster, dog, monkey, and man. 1966 May. Cancer Chemother. Reports 50(4): 219-244. PMID: 4957125).

The ASOs described above may be administered in a pharmaceutical composition comprising therapeutically effective amounts of an ASO together with pharmaceutically acceptable excipients, diluents, preservatives, solubilizers, emulsifiers, adjuvants and/or carriers. Such compositions include diluents of various buffer content (e.g., Tris-HCl, acetate, phosphate), pH and ionic strength and additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol). The material may be incorporated into particulate preparations of polymeric compounds such as polylactic acid, polyglycolic acid, etc. or into liposomes. Hylauronic acid may also be used. Such compositions may influence the physical state, stability, rate of in vivo release, and rate of in vivo clearance of the present proteins and derivatives. The compositions may be prepared in liquid form, or may be in dried powder, such as lyophilised form.

### Administration

A pharmaceutical composition comprising an ASO and a pharmaceutically acceptable carrier or excipient may be prepared for administration according to techniques well known in the pharmaceutical industry. Such techniques include, but are not limited to, combining the ASO with the carrier and/or excipient(s) into association in a unit dosage form.

Compositions suitable for oral administration may be presented in discrete units, such as capsules, cachets, lozenges, or tablets, each containing a predetermined amount of a compound of the present disclosure as powder or granules; as a solution or a suspension in an aqueous or non-aqueous liquid; or as an oil-in-water or water-in-oil emulsion. As indicated, such formulations may be prepared by any suitable method of pharmacy which includes the step of bringing into association at least one embodiment of the present disclosure as the active compound and a carrier or excipient (which may constitute one or more accessory ingredients). The carrier must be acceptable in the sense of being compatible with the other ingredients of the formulation and must not be deleterious to the recipient. The carrier may be a solid or a liquid, or both, and may be formulated with at least one compound described herein as the active compound in a unit-dose formulation, for example, a tablet, which may contain from about 0.05% to about 95% by weight of the at least one active compound. Other pharmacologically active substances may also be present including other compounds. The formulations of the present disclosure may be prepared by any of the well-known techniques of pharmacy consisting essentially of admixing the components.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by, for example, dissolving or dispersing, at least one active compound of the present disclosure as described herein and optional pharmaceutical adjuvants in an excipient, such as, for example, water, saline, aqueous dextrose, glycerol, ethanol, and the like, to thereby form a solution or suspension. In general, suitable formulations may be prepared by uniformly and intimately admixing the at least one active compound of the present disclosure with a liquid or finely divided solid carrier, or both, and then, if necessary, shaping the product. For example, a tablet may be prepared by compressing or molding a powder or granules of at least one embodiment of the present disclosure, which may be optionally combined with one or more accessory ingredients. Compressed tablets may be prepared by compressing, in a suitable machine, at least one embodiment of the present disclosure in a free-flowing form, such as a powder or granules, which may be optionally mixed with a binder, lubricant, inert diluent and/or surface active/dispersing agent(s). Molded tablets may be made by molding, in a suitable machine, where the powdered form of at least one embodiment of the present disclosure is moistened with an inert liquid diluent.

Formulations suitable for buccal (sub-lingual) administration include lozenges comprising at least one embodiment of the present disclosure in a flavored base, usually sucrose and acacia or tragacanth, and pastilles comprising the at least one compound in an inert base such as gelatin and glycerin or sucrose and acacia.

Formulations suitable for parenteral administration comprise sterile aqueous preparations of at least one embodiment of the present disclosure, which are approximately isotonic with the blood of the intended recipient. These preparations are administered intravenously, although administration may also be affected by means of subcutaneous, intramuscular, intraperitoneal, intracerebroventricular, or intradermal injection. Such preparations may conveniently be prepared by admixing at least one embodiment described herein with water and rendering the resulting solution sterile and isotonic with the blood. Injectable compositions according to the present disclosure may contain from about 0.1 to about 5% w/w of the active compound.

Formulations suitable for rectal administration are presented as unit-dose suppositories. These may be prepared by admixing at least one embodiment as described herein with one or more conventional solid carriers, for example, cocoa butter, and then shaping the resulting mixture.

Formulations suitable for topical application to the skin may take the form of an ointment, cream, lotion, paste, gel, spray, aerosol, or oil. Carriers and excipients which may be used include Vaseline, lanoline, polyethylene glycols, alcohols, and combinations of two or more thereof. The ASO is generally present at a concentration of from about 0.1% to about 15% w/w of the composition, for example, from about 0.5 to about 2%.

### EXAMPLES

The following Example serves to more fully describe the disclosure. It is meant for illustrative purposes and is not meant to limit the disclosure in any way.

### Example 1—Preparation and Screening of ASOs

### Materials and Methods

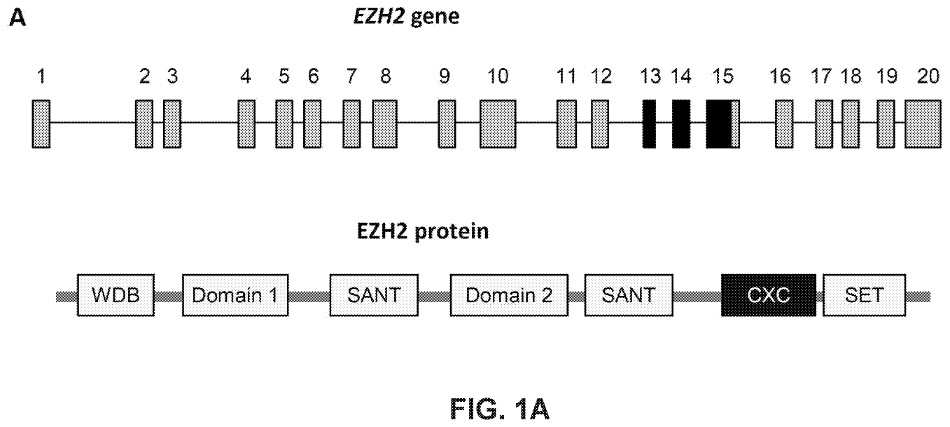

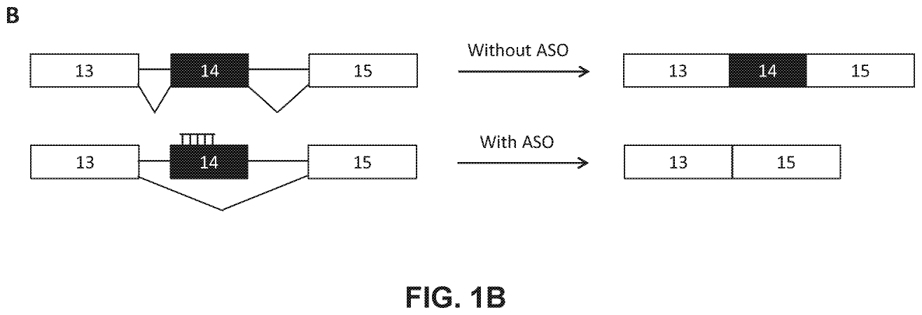

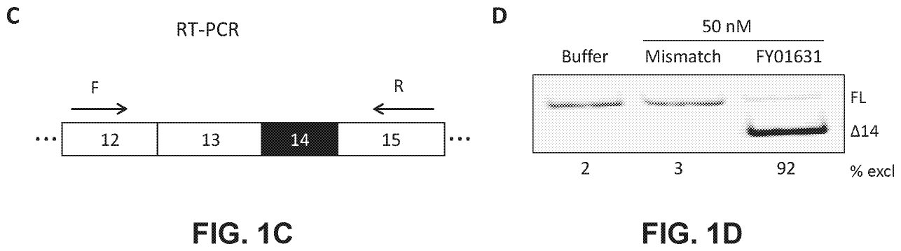

ASOs modified with 2′-O-methoxyethyl (MOE) ribose, a phosphorothioate (PS) backbone, and containing 5-methylcytosines in place of all cytosines were purchased from Biosyntech (Suzhou, China) and dissolved in DEPC-treated water with a stock solution at 20 μM. The EZH2 gene and EZH2 protein domain structures are shown in FIGS. 1A-1B. The EZH2 gene comprises of 20 exons and EZH2 protein contains multiple domains including a WDB (EED-binding) domain, Domain 1, Domain 2, two SANT (Swi3, Ada2, N-Cir, and TFIIIB) domains, a CXC (cysteine-rich) domain and a SET (catalytic) domain. Effective ASOs targeting two regions of exon 14 are shown in Table 1. The effects of ASO FY01631 and a mismatch ASO (Mismatch) on EZH2 exon 14 splicing were investigated; buffer (without adding ASO) was used as an additional negative control. ASO FY01631 and Mismatch were respectively transfected into a MHCC97H human hepatocellular carcinoma cell line using Exfect Transfection Reagent (Vazyme Biotech, China), and splicing was analyzed by semiquantitative fluorescent RT-PCR and native PAGE. ASO FY01631 represents an optimal ASO that was obtained from the screening method described herein. Mismatch (SEQ ID NO:19) is a mismatch version of FY01631, containing 8 mismatched nucleotides compared to FY01631 (see Table 1). FY01631 promoted exon 14 skipping, whereas Mismatch had no effect (FIG. 1D).

The ASO screening method described herein included three steps:
1) an initial first-round screening involving a series of overlapping 20-mers to identify potential target regions; in the case of EZH2 exon 14, two potential target regions were identified—one around the target sequence of FY01604 and the other around the target sequence of FY01606;
2) a further high-resolution ASO screening to identify optimal ASOs; this step was also called ASO microwalk. ASOs were positioned in 1-nt steps along the potential target regions identified in the initial screening; and
3) a final length optimization of the optimal ASOs identified in the high-resolution ASO screening to obtain lead ASOs.

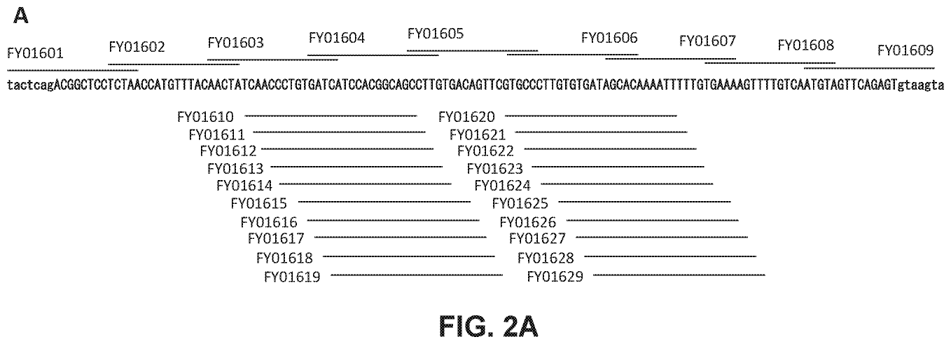

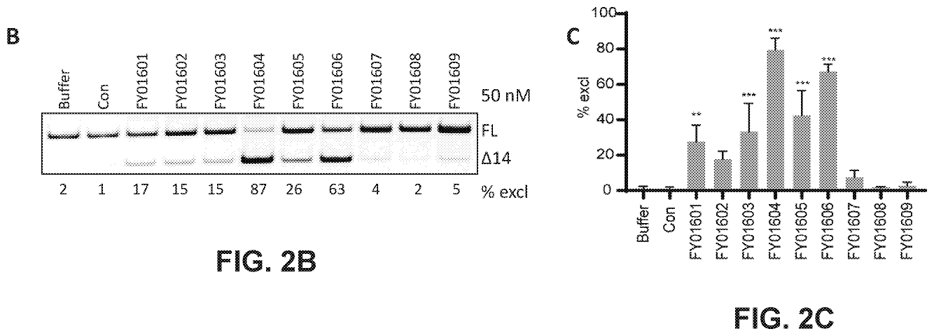

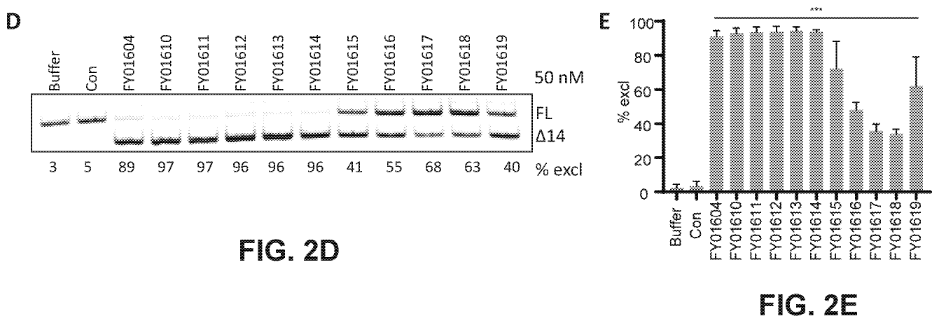

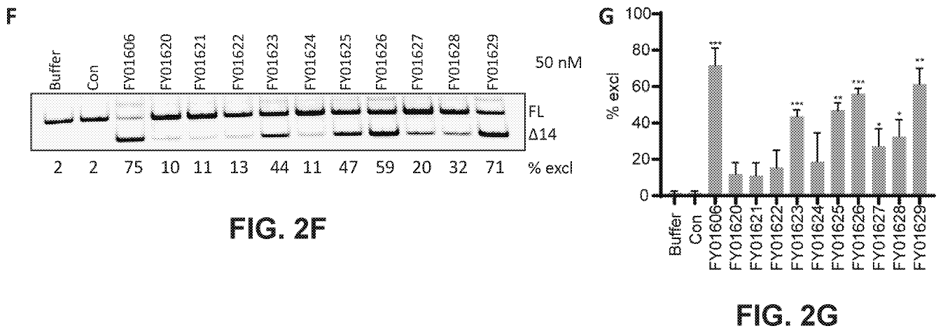

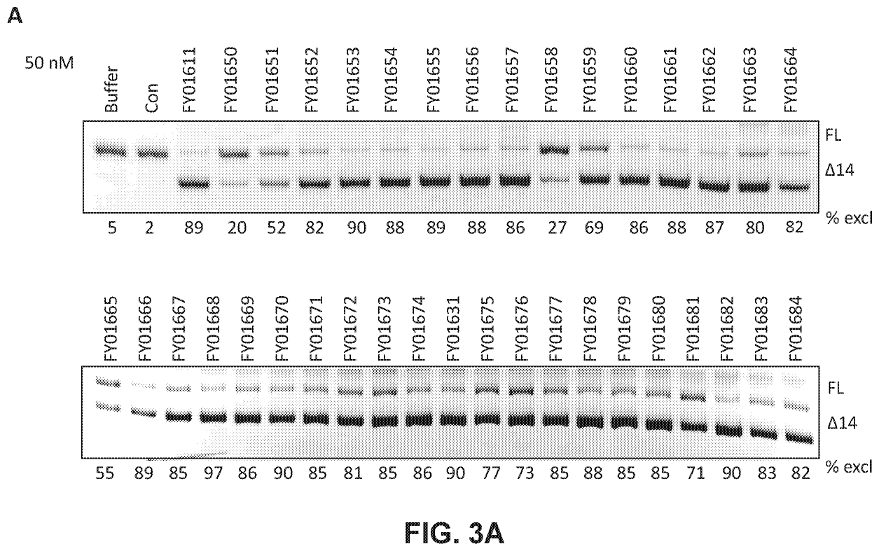

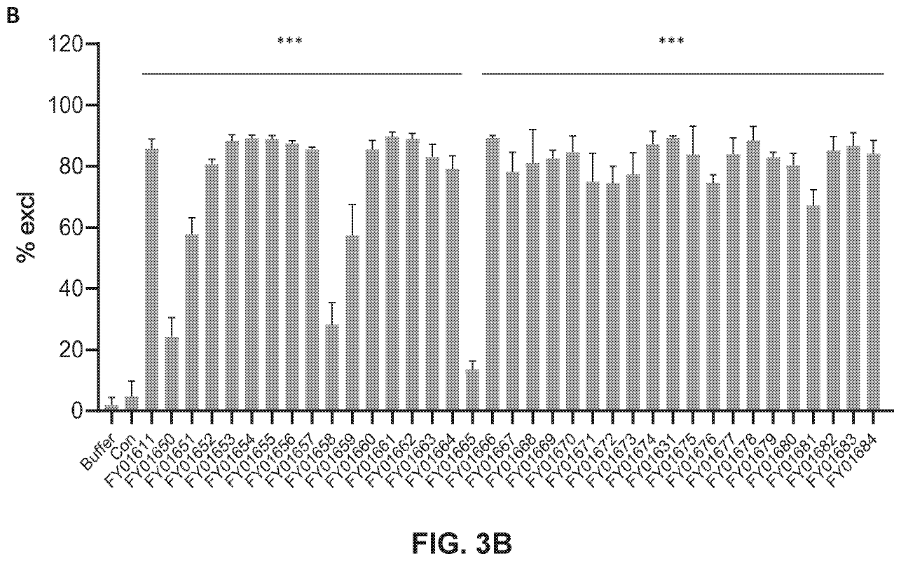

All ASOs tested in the Examples section are shown in Tables 1 and 2. FIG. 2A shows alignments of the ASOs tested in the ASO screening experiments described herein. The EZH2 exon 14 sequence is shown in uppercase and flanking intron sequences in lowercase. In the initial first-round screening of ASOs, nine 20-nt ASOs (indicated by horizontal bars above their respective target nucleotide sequence in FIG. 2A) were used, with 5-nt overlap between each two adjacent ASOs as shown in FIG. 2A. In the further microwalk screening of ASOs, a total of twenty 20-nt ASOs (indicated by bars below the target sequence in FIG. 2A) were used, with 1-nt steps between each two adjacent ASOs. In the final investigation of the effect of different ASO lengths on exon 14 alternative splicing, a total of 35 additional ASOs from 21- to 28-nt long were tested (see Table 2 and FIGS. 3A-3B). Each ASO was transfected into MHCC97H cells at a concentration of 50 nM. Buffer and an unrelated scrambled ASO (5′-TTGTATTCTATGTTT-3′) (Con) were used as controls. After 30 h of transfection, RT-PCR was performed using Cy5-labelled primers and the products were subsequently separated by native PAGE. FIG. 2C shows the quantitation of the ASO initial screening in FIG. 2B, and two more repeated independent experiments of same. FIGS. 2D and 2F show high-resolution screening performed for the two best performing ASOs (FY01604 and FY01606) from the initial screening as shown in FIG. 2B. Quantitation of the further screening in FIGS. 2D and 2F was conducted and shown in FIGS. 2E and 2G. Multiple potent ASOs were identified in the final length optimization step as shown FIGS. 3A-3B and Table 2. One representative of three independent experiments was shown. FL: full length; Δ14: transcript with exon 14 skipping. For all statistics, n=3, *P<0.05, **P<0.01, ***P<0.001 versus buffer.

EZH2 exon 14 (uppercase) and flanking intron sequences (lowercase) are: <cctgcatgatgttatctttactcagACGGCTCCTCTAACCATGTTTACAACTATCAACCCTGTGATCA TCCACGGCAGCCTTGTGACAGTTCGTGCCCTTGTGTGATAGCACAAAATTTTTGTGA AAAGTTTTGTCAATGTAGTTCAGAGTgtaagtatttgttgctttgatgcaa (SEQ ID NO:22)>.

A large number of ASOs targeting exon 14 and flanking intron sequences were designed and screened with the endogenous EZH2 gene in MHCC97H cells. Multiple ASOs targeting a 28-nt sequence from position 31 to position 58 or another 27-nt sequence from position 67 to position 93 in exon 14 markedly promoted exon 14 skipping in cultured cells. ASO sequences and effects in inducing exon 14 skipping are shown in Tables 1 and 2. The two underlined regions (see above) in exon 14 are preferred ASO targets.

TABLE 1
ASOs tested in Example 1 and shown in FIGS. 1A to 2G. All ASOs are MOE-
modified with phosphorothioate backbone and all 5-methyl cytosines.

| | | | | | Effect in |
|---|---|---|---|---|---|
| | | | | | inducing |
| SEQ ID | ASO | Sequence | Length | Target in EZH2 pre-mRNA | exon |
| NO | name | (5′-3′) | (nt) | (5′-3′) | skipping |
| SEQ ID | FY01631 | CAAGGCTGCCGTGGATGAT | 24 | CUGUGAUCA- | +++++ |
| NO: 1 | | CACAG | | UCCACGGCAGCCUUG | |
| SEQ ID | FY01610 | CTGCCGTGGATGATCACAG | 20 | CCUGUGAUCAUCCACGGC | ++++ |
| NO: 2 | | G | | AG | |
| SEQ ID | FY01611 | GCTGCCGTGGATGATCACA | 20 | CUGUGAUCAUCCACGGCA | ++++ |
| NO: 3 | | G | | GC | |
| SEQ ID | FY01612 | GGCTGCCGTGGATGATCAC | 20 | UGUGAUCAUCCACGGCAG | ++++ |
| NO: 4 | | A | | CC | |
| SEQ ID | FY01613 | AGGCTGCCGTGGATGATCA | 20 | GUGAUCAUCCACGGCAGC | ++++ |
| NO: 5 | | C | | CU | |
| SEQ ID | FY01614 | AAGGCTGCCGTGGATGATC | 20 | UGAUCAUCCACGGCAGCC | ++++ |
| NO: 6 | | A | | UU | |
| SEQ ID | FY01604 | CAAGGCTGCCGTGGATGAT | 20 | GAUCAUCCACGGCAGCCU | +++ |
| NO: 7 | | C | | UG | |
| SEQ ID | FY01615 | ACAAGGCTGCCGTGGATGA | 20 | AUCAUCCACGGCAGCCUU | +++ |
| NO: 8 | | T | | GU | |
| SEQ ID | FY01616 | CACAAGGCTGCCGTGGATG | 20 | UCAUCCACGGCAGCCUUG | ++ |
| NO: 9 | | A | | UG | |
| SEQ ID | FY01617 | TCACAAGGCTGCCGTGGAT | 20 | CAUCCACGGCAGCCUUGU | ++ |
| NO: 10 | | G | | GA | |
| SEQ ID | FY01618 | GTCACAAGGCTGCCGTGGA | 20 | AUCCACGGCAGCCUUGUG | ++ |
| NO: 11 | | T | | AC | |
| SEQ ID | FY01619 | TGTCACAAGGCTGCCGTGG | 20 | UCCACGGCAGCCUUGUGA | ++ |
| NO: 12 | | A | | CA | |
| SEQ ID | FY01623 | TGCTATCACACAAGGGCAC | 20 | CGUGCCCUUGUGUGAUAG | ++ |
| NO: 13 | | G | | CA | |
| SEQ ID | FY01606 | TGTGCTATCACACAAGGGC | 20 | UGCCCUUGUGUGAUAGCA | ++ |
| NO: 14 | | A | | CA | |
| SEQ ID | FY01625 | TTGTGCTATCACACAAGGG | 20 | GCCCUUGUGUGAUAGCAC | ++ |
| NO: 15 | | C | | AA | |
| SEQ ID | FY01626 | TTTGTGCTATCACACAAGG | 20 | CCCUUGUGUGAUAGCACA | ++ |
| NO: 16 | | G | | AA | |
| SEQ ID | FY01628 | ATTTTGTGCTATCACACAA | 20 | CUUGUGUGAUAGCACAAA | + |
| NO: 17 | | G | | AU | |
| SEQ ID | FY01629 | AATTTTGTGCTATCACACA | 20 | UUGUGUGAUAGCACAAAA | +++ |
| NO: 18 | | A | | UU | |
| SEQ ID | Mismatch | CATGGGTGTCGCGGGTGGT | 24 | GUGAGACCACCCGCGACA | |
| NO: 19 | | CTCAC | | CCCAUG | |
| SEQ ID | FY01601 | TTAGAGGAGCCGTCTGAGT | 20 | UACUCAGACGGCUCCUCU | + |
| NO: 20 | | A | | AA | |
| SEQ ID | FY01602 | TAGTTGTAAACATGGTTAG | 20 | UCUAACCAUGUUUACAAC | + |
| NO: 21* | | A | | UA | |
| SEQ ID | FY01603 | TGATCACAGGGTTGATAGT | 20 | AACUAUCAACCCUGUGAU | + |
| NO: 23 | | T | | CA | |
| SEQ ID | FY01605 | GGGCACGAACTGTCACAAG | 20 | CCUUGUGACAGUUCGUGC | ++ |
| NO: 24 | | G | | CC | |
| SEQ ID | FY01607 | TTTTCACAAAAATTTTGTGC | 20 | GCACAAAAUUUUUGUGAA | / |
| NO: 25 | | | | AA | |
| SEQ ID | FY01608 | CTACATTGACAAAACTTTT | 20 | GAAAAGUUUUGUCAAUGU | / |
| NO: 26 | | C | | AG | |
| SEQ ID | FY01609 | TACTTACACTCTGAACTAC | 20 | UGUAGUUCAGAGUGUAAG | / |
| NO: 27 | | A | | UA | |
| SEQ ID | FY01620 | TATCACACAAGGGCACGAA | 20 | GUUCGUGCCCUUGUGUGA | / |
| NO: 28 | | C | | UA | |
| SEQ ID | FY01621 | CTATCACACAAGGGCACGA | 20 | UUCGUGCCCUUGUGUGAU | / |
| NO: 29 | | A | | AG | |
| SEQ ID | FY01622 | GCTATCACACAAGGGCACG | 20 | UCGUGCCCUUGUGUGAUA | / |
| NO: 30 | | A | | GC | |
| SEQ ID | FY01624 | GTGCTATCACACAAGGGCA | 20 | GUGCCCUUGUGUGAUAGC | / |
| NO: 31 | | C | | AC | |
| SEQ ID | FY01627 | TTTTGTGCTATCACACAAG | 20 | CCUUGUGUGAUAGCACAA | + |
| NO: 32 | | G | | AA | |

*: SEQ ID NO: 19, also referred to as Mismatch, is an ASO containing 8 mismatched nucleotides compared to FY01631 (SEQ ID NO: 1).

TABLE 2
Additional 35 ASOs tested as described in Example 1 and shown in FIGS. 3A-3B.

| | ASO | | Length | |
|---|---|---|---|---|
| SEQ ID NO | name | Sequence (5′-3′) | (nt) | Effect |
| SEQ ID | FY01650 | GCCGTGGATGATCACAGGGTT | 21 | + |
| NO: 33 | | | | |
| SEQ ID | FY01651 | TGCCGTGGATGATCACAGGGT | 21 | + |
| NO: 34 | | | | |
| SEQ ID | FY01652 | CTGCCGTGGATGATCACAGGG | 21 | ++++ |
| NO: 35 | | | | |
| SEQ ID | FY01653 | GCTGCCGTGGATGATCACAGG | 21 | +++++ |
| NO: 36 | | | | |
| SEQ ID | FY01654 | GGCTGCCGTGGATGATCACAG | 21 | ++++ |
| NO: 37 | | | | |
| SEQ ID | FY01655 | AGGCTGCCGTGGATGATCACA | 21 | +++ |
| NO: 38 | | | | |
| SEQ ID | FY01656 | AAGGCTGCCGTGGATGATCAC | 21 | +++ |
| NO: 39 | | | | |
| SEQ ID | FY01657 | CAAGGCTGCCGTGGATGATCA | 21 | ++++ |
| NO: 40 | | | | |
| SEQ ID | FY01658 | TGCCGTGGATGATCACAGGGTT | 22 | + |
| NO: 41 | | | | |
| SEQ ID | FY01659 | CTGCCGTGGATGATCACAGGGT | 22 | ++ |
| NO: 42 | | | | |
| SEQ ID | FY01660 | GCTGCCGTGGATGATCACAGGG | 22 | +++++ |
| NO: 43 | | | | |
| SEQ ID | FY01661 | GGCTGCCGTGGATGATCACAGG | 22 | ++++ |
| NO: 44 | | | | |
| SEQ ID | FY01662 | AGGCTGCCGTGGATGATCACAG | 22 | ++++ |
| NO: 45 | | | | |
| SEQ ID | FY01663 | AAGGCTGCCGTGGATGATCACA | 22 | +++ |
| NO: 46 | | | | |
| SEQ ID | FY01664 | CAAGGCTGCCGTGGATGATCAC | 22 | +++ |
| NO: 47 | | | | |
| SEQ ID | FY01665 | CTGCCGTGGATGATCACAGGGTT | 23 | − |
| NO: 48 | | | | |
| SEQ ID | FY01666 | GCTGCCGTGGATGATCACAGGGT | 23 | ++++ |
| NO: 49 | | | | |
| SEQ ID | FY01667 | GGCTGCCGTGGATGATCACAGGG | 23 | ++++ |
| NO: 50 | | | | |
| SEQ ID | FY01668 | AGGCTGCCGTGGATGATCACAGG | 23 | +++ |
| NO: 51 | | | | |
| SEQ ID | FY01669 | AAGGCTGCCGTGGATGATCACAG | 23 | +++ |
| NO: 52 | | | | |
| SEQ ID | FY01670 | CAAGGCTGCCGTGGATGATCACA | 23 | ++++ |
| NO: 53 | | | | |
| SEQ ID | FY01671 | GCTGCCGTGGATGATCACAGGGTT | 24 | ++++ |
| NO: 54 | | | | |
| SEQ ID | FY01672 | GGCTGCCGTGGATGATCACAGGGT | 24 | +++++ |
| NO: 55 | | | | |
| SEQ ID | FY01673 | AGGCTGCCGTGGATGATCACAGGG | 24 | ++ |
| NO: 56 | | | | |
| SEQ ID | FY01674 | AAGGCTGCCGTGGATGATCACAGG | 24 | +++ |
| NO: 57 | | | | |
| SEQ ID | FY01675 | GGCTGCCGTGGATGATCACAGGGTT | 25 | ++++ |
| NO: 58 | | | | |
| SEQ ID | FY01676 | AGGCTGCCGTGGATGATCACAGGGT | 25 | +++++ |
| NO: 59 | | | | |
| SEQ ID | FY01677 | AAGGCTGCCGTGGATGATCACAGGG | 25 | +++++ |
| NO: 60 | | | | |
| SEQ ID | FY01678 | CAAGGCTGCCGTGGATGATCACAGG | 25 | +++ |
| NO: 61 | | | | |
| SEQ ID | FY01679 | AGGCTGCCGTGGATGATCACAGGGTT | 26 | +++ |
| NO: 62 | | | | |
| SEQ ID | FY01680 | AAGGCTGCCGTGGATGATCACAGGGT | 26 | +++ |
| NO: 63 | | | | |
| SEQ ID | FY01681 | CAAGGCTGCCGTGGATGATCACAGGG | 26 | +++ |
| NO: 64 | | | | |
| SEQ ID | FY01682 | AAGGCTGCCGTGGATGATCACAGGGTT | 27 | +++ |
| NO: 65 | | | | |
| SEQ ID | FY01683 | CAAGGCTGCCGTGGATGATCACAGGGT | 27 | +++ |
| NO: 66 | | | | |
| SEQ ID | FY01684 | CAAGGCTGCCGTGGATGATCACAGGGT | 28 | ++++ |
| NO: 67 | | T | | |

### Example 2—Growth Inhibition Effect of FY01631 in HCC Cells

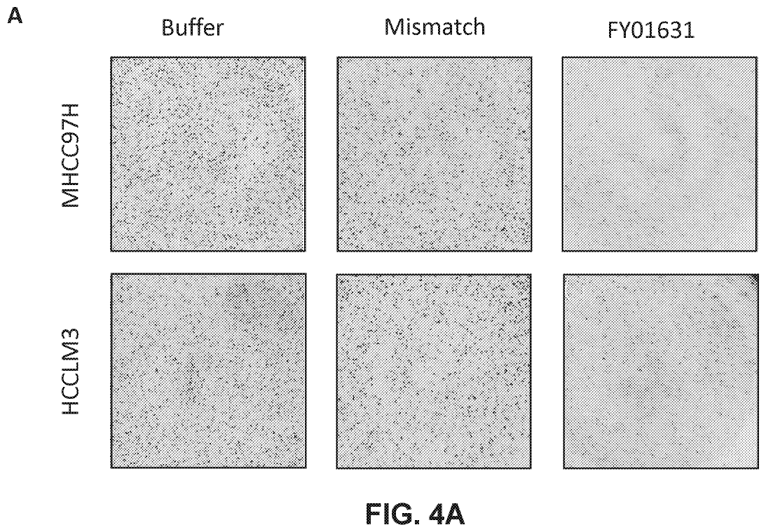

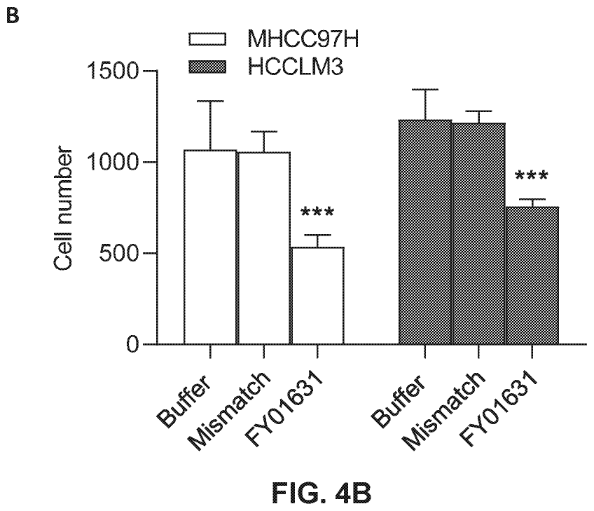

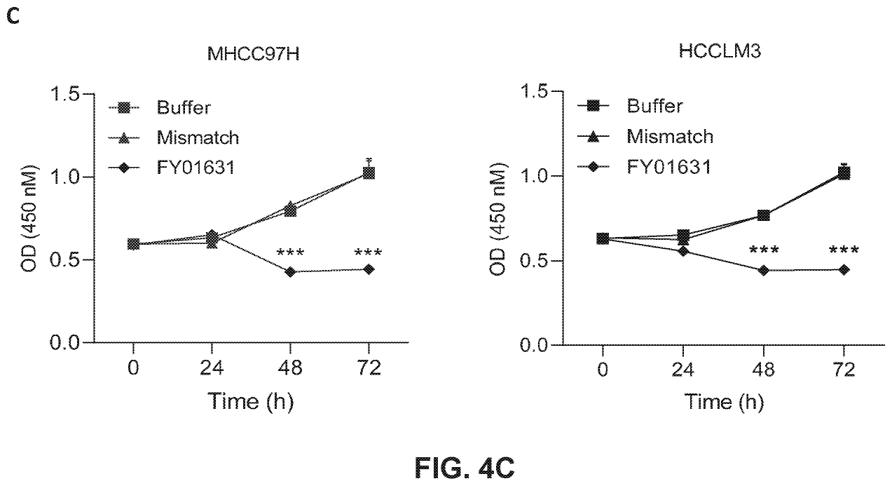

Hepatocellular carcinoma cell lines MHCC97H and HCCLM3 were transfected with FY01631 (50 nM), Mismatch (50 nM), or buffer and incubated for 48 hours; then 5,000 cells from each treatment group were respectively seeded into culture dishes and incubated for 10 days, with fresh medium being changed every 48 hours. Cells were then stained using crystal violet solution and counted. FY01631 markedly inhibited tumor cell proliferation (FIGS. 4A-4B). Live cells were quantitated by the tetrazolium assay. Cell Counting Kit-8 was used on HCC cells (MHCC97H and HCCLM3 cells in 96-well plates) after transfection with FY01631 (50 nM), Mismatch (50 nM) or buffer and subsequent incubation for 0, 24, 48 or 72 hours (h). Absorbance was measured at 450 nm by a microplate reader. Cell Counting kit-8 results showed that FY01631 inhibited the growth of MHCC97H and HCCLM3 cells when post-transfection incubation time reached 48 hours (FIG. 4C).

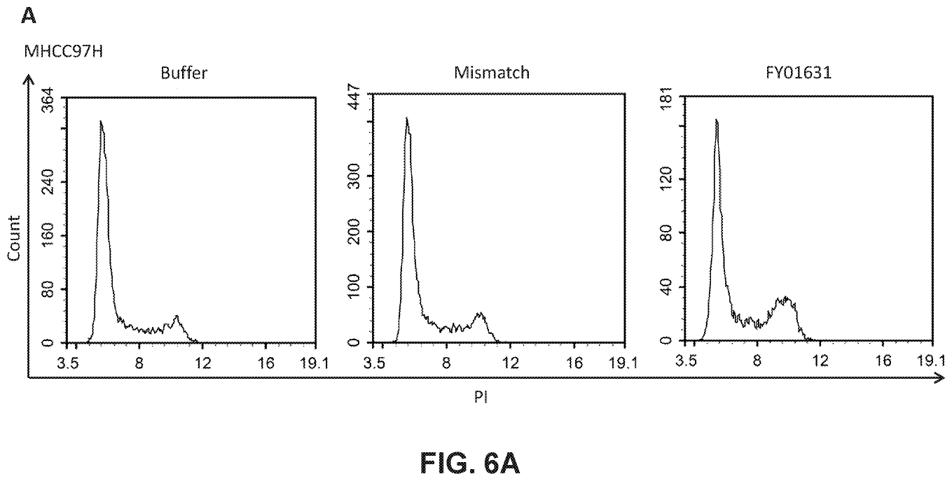

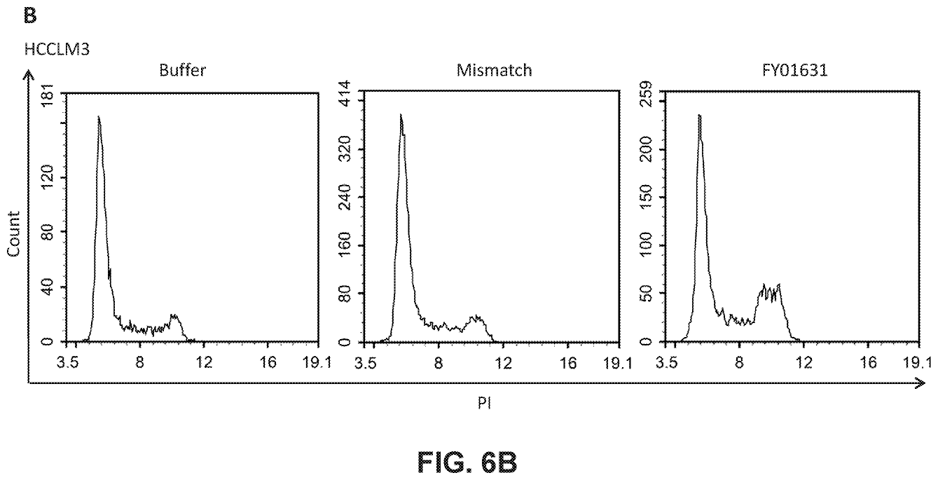

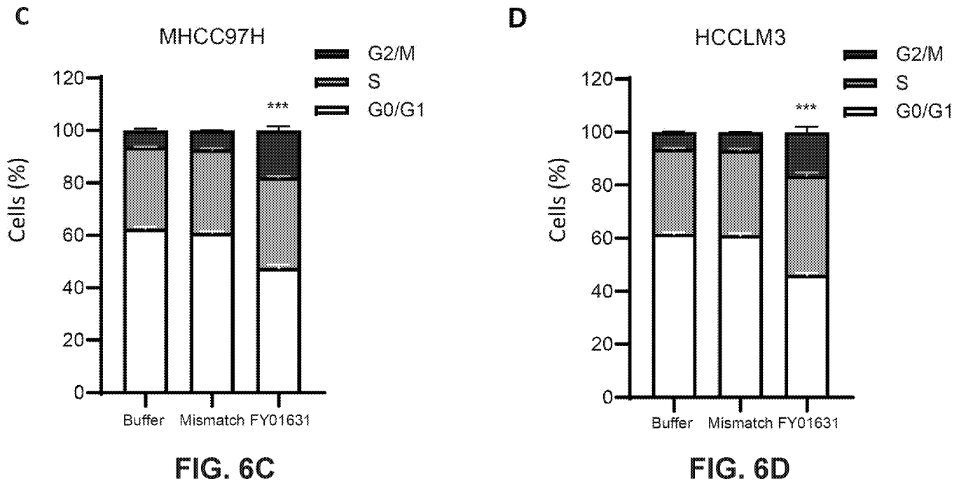

48 hours post transfection with FY01631 (50 nM), Mismatch (50 nM), or buffer alone, propidium iodine (PI) staining-coupled flow cytometric assays were conducted and showed that FY01631 significantly promoted the accumulation of both hepatocellular carcinoma MHCC97H and HCCLM3 cells in the G2/M phase (FIGS. 6A-6D). Buffer: without adding ASO; Mismatch: control ASO. For all statistics, n=3, *P<0.05, **P<0.01, *6 **P<0.001 versus buffer.

### Example 3—Promoted Apoptosis of Tumor Cells by FY01631

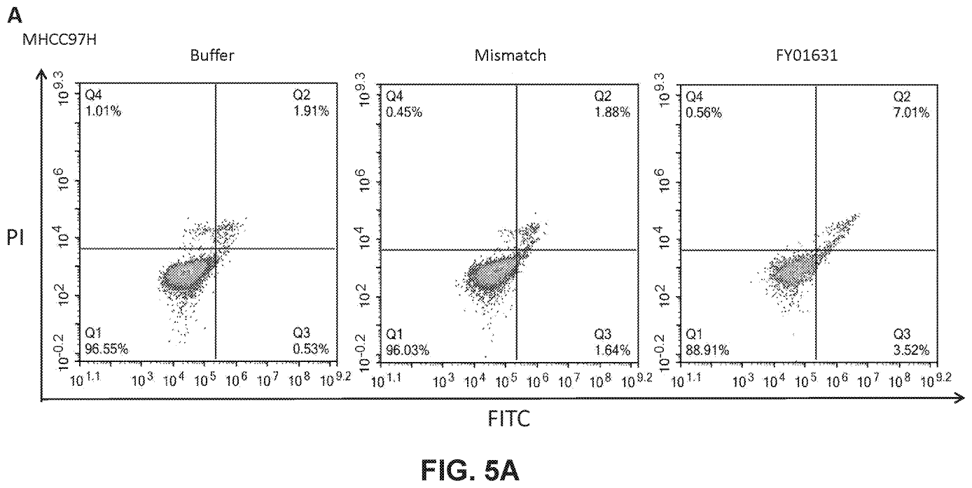

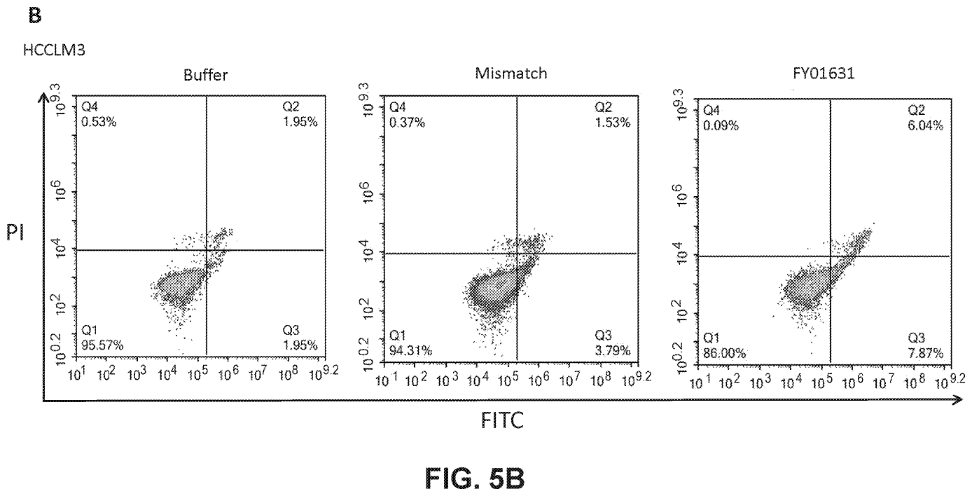

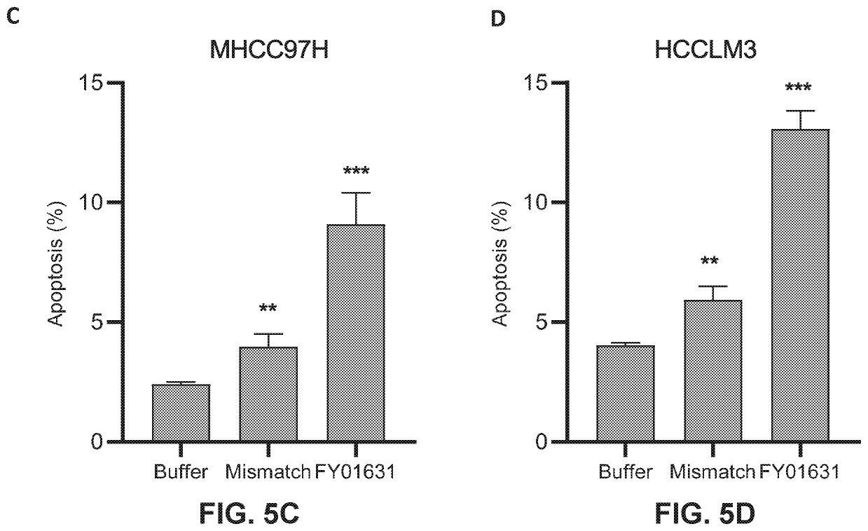

MHCC97H and HCCLM3 were transfected with FY01631, Mismatch, or buffer and incubated for 48 hours. Cells were harvested and stained with propidium iodide (PI) and FITC-Annexin V (Transgene Biotech, Beijing, China), and then subjected to flow cytometric sorting to determine the percentage of apoptotic cells, FIG. 5A: MHCC97H and FIG. 5B: HCCLM3. As shown in FIGS. 5C and 5D, FY01631 markedly promoted apoptosis of tumor cells. For all groups, n=3; *P<0.05, ***P<0.001 versus buffer.

### Example 4—Promoted EZH2 Exon 14 Skipping and Downregulated H3K27Me3 Expression In Vivo by FY01631

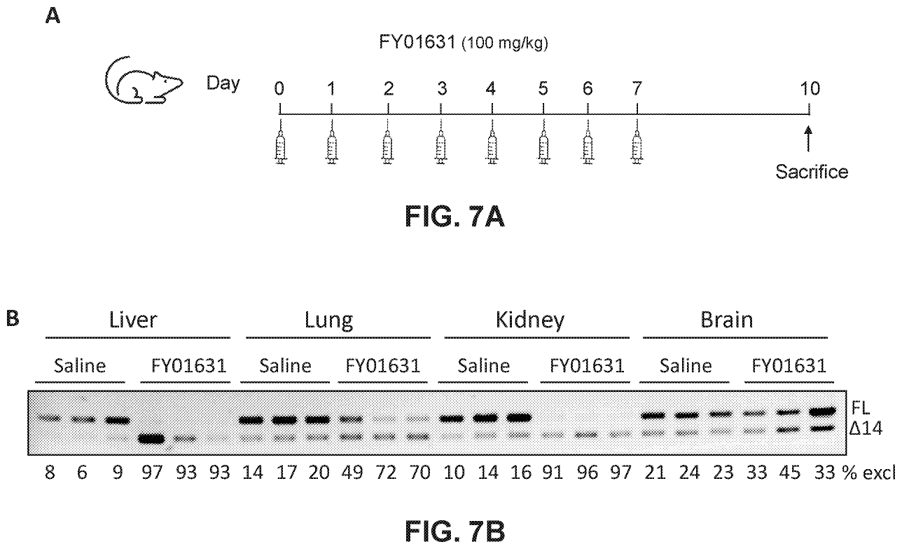

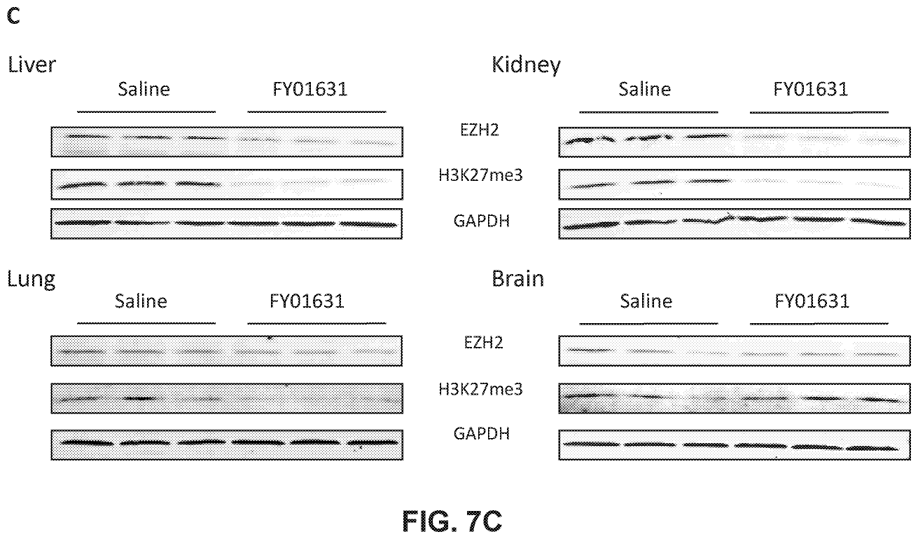

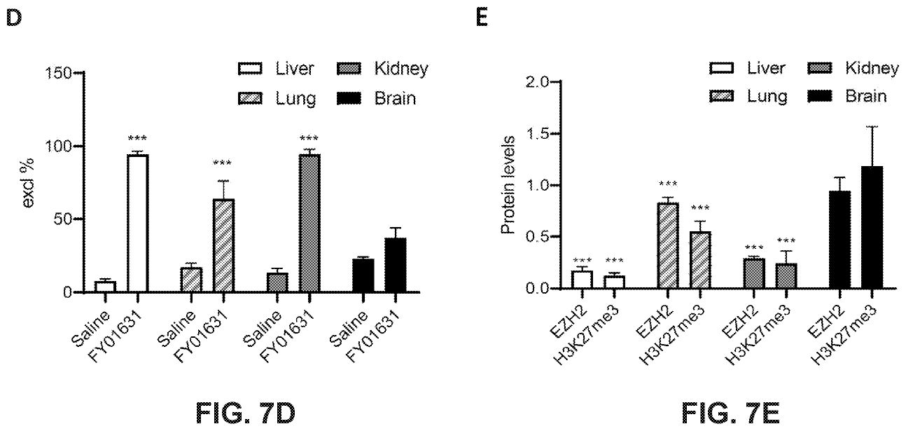

Six-week-old BALB/c mice (n=3 for each group) were injected subcutaneously with FY01631 at 100 μg/g per day for eight consecutive days, and saline was used as negative control. Euthanasia was performed 3 days after the last injection (FIG. 7A). Tissue samples such as liver, lung, kidney and brain were collected for RT-PCR analysis (FIG. 7B). Strong effect of mouse Ezh2 exon 14 skipping was observed in liver and kidney samples, and moderate effect was observed in lung, whereas no effect was observed in brain samples, as shown in FIG. 7B.

Western blotting analysis were performed for EZH2 and its downstream protein H3K27me3 in the liver, lung, kidney, and brain samples; GAPDH was used as loading control. Expression levels of both proteins were strikingly decreased in liver and kidney, and moderately in lung after FY01631 treatment compared to saline control, as shown in FIG. 7C. Splicing data from three independent experiments were quantitated and shown in FIG. 7D. Data from three independent data were quantitated and shown in FIG. 7E. All protein levels were first normalized to GAPDH and then normalized to saline control with the protein levels in saline group being set as 1. For all groups, n=3; ***P<0.001 versus saline.

### Example 5—Inhibited Tumor Growth in Xenograft Mouse Model

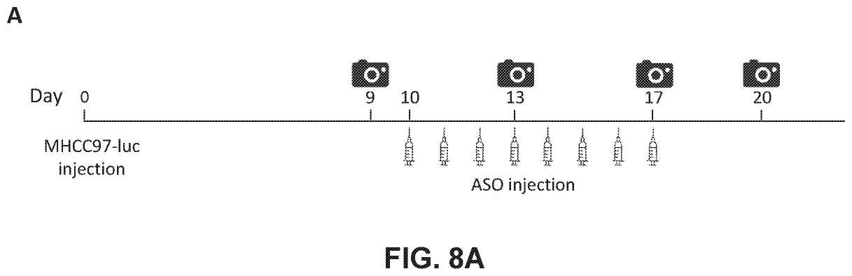

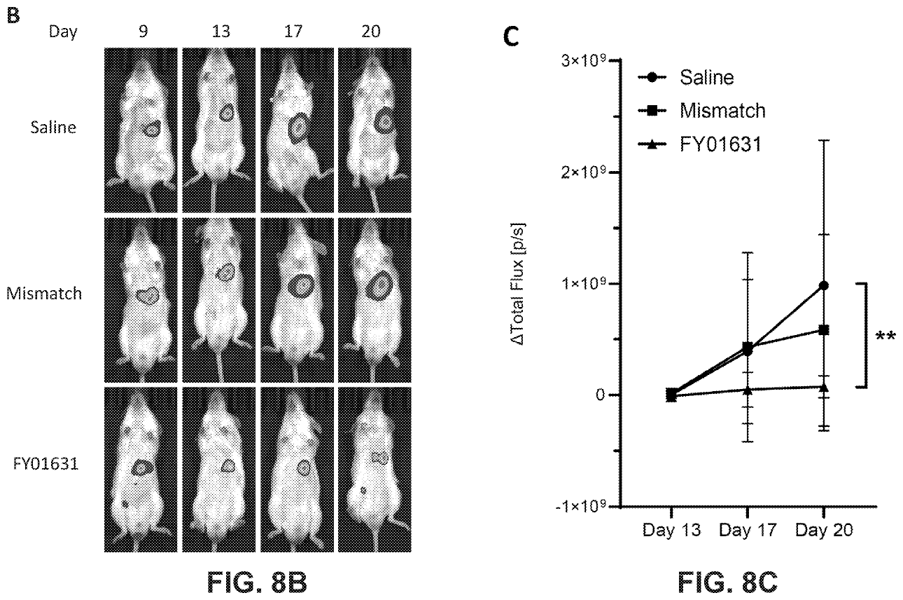

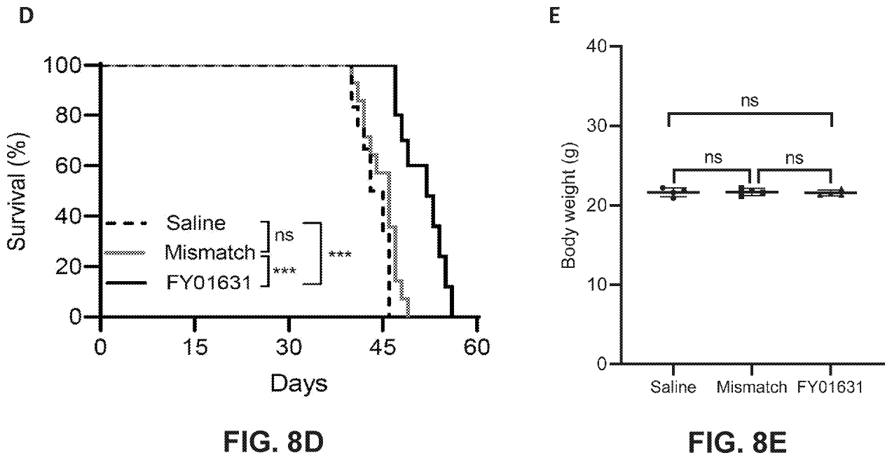

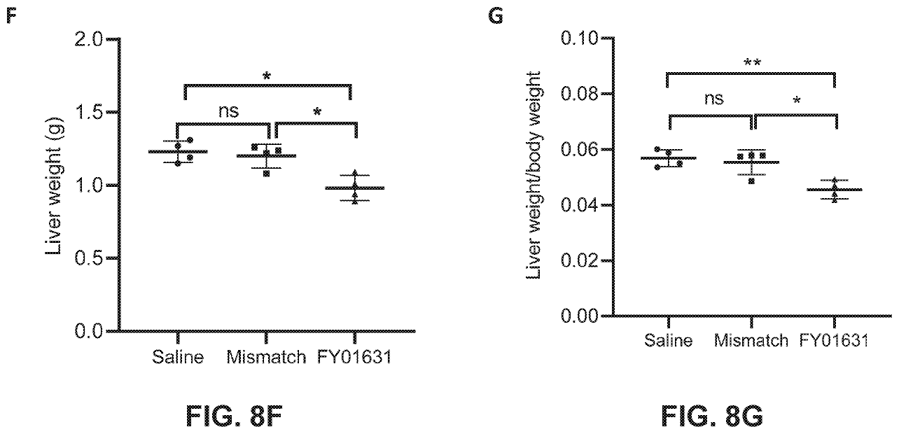

1×10⁶ luciferase-integrated MHCC97H cells were inoculated in situ into NOD/SCID mice at 6 weeks of age and the inoculation date was set as day 0. Tumors were allowed to grow for 10 days, and then mice were dosed subcutaneously with FY01631 at 100 mg/kg per day for 8 consecutive days; Mismatch and saline were used as controls. A total of 48 mice were randomly divided into 3 groups with each group having 16 mice. Whole-body live imaging of mice was performed on days 9, 13, 17 and 20. Mice were given 150 mg/kg D-luciferin by intraperitoneal injection 10 minutes prior to imaging. Among the 16 mice for each group, 12 were preset for evaluation of survival and 4 were preset for evaluation of liver weight and tumor histological changes. Mice treated with FY01631 (dark solid line) had much longer survival compared to other two groups, the saline group (dashed line) and the Mismatch group (light grey solid line) (FIG. 8D). The body weight, liver weight, and ratio of liver weight to body weight of the preset four mice were recorded at the time of sacrifice immediately after the final imaging. Liver weight and ratio of liver weight to body weight were significantly reduced in mice treated with FY01631 compared to saline and Mismatch, as shown in FIGS. 8E-8F. *P<0.05, **P<0.01; ns: no significance. Therefore, FY01631 markedly inhibited liver tumor growth in a xenograft mouse model.

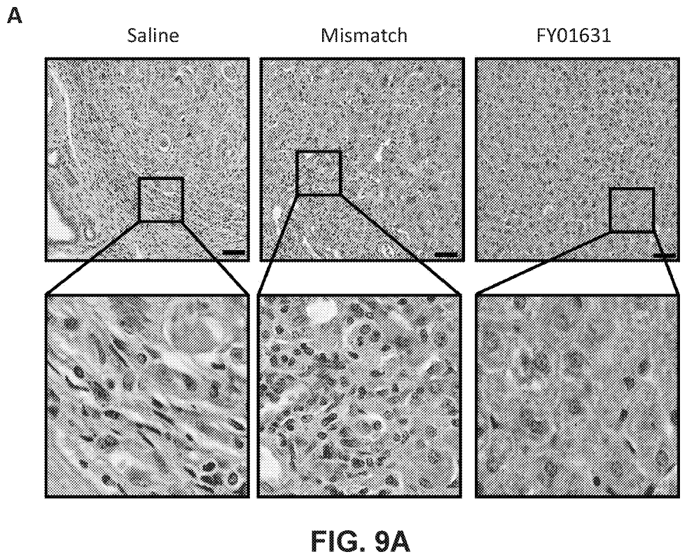

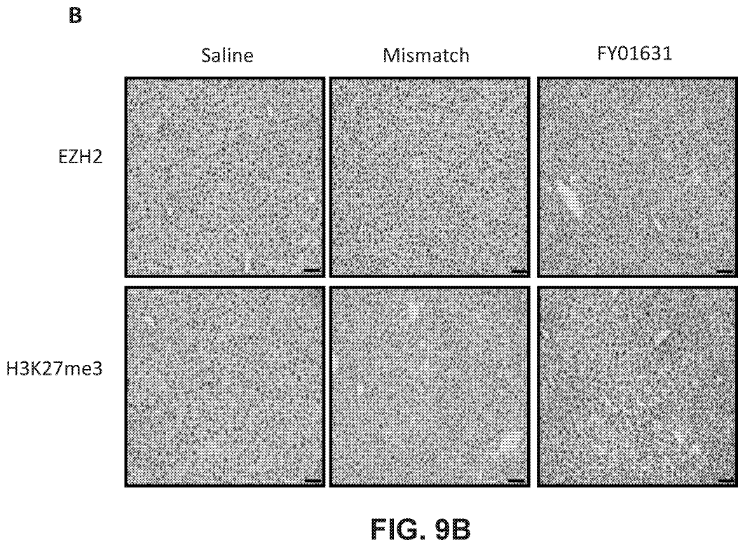

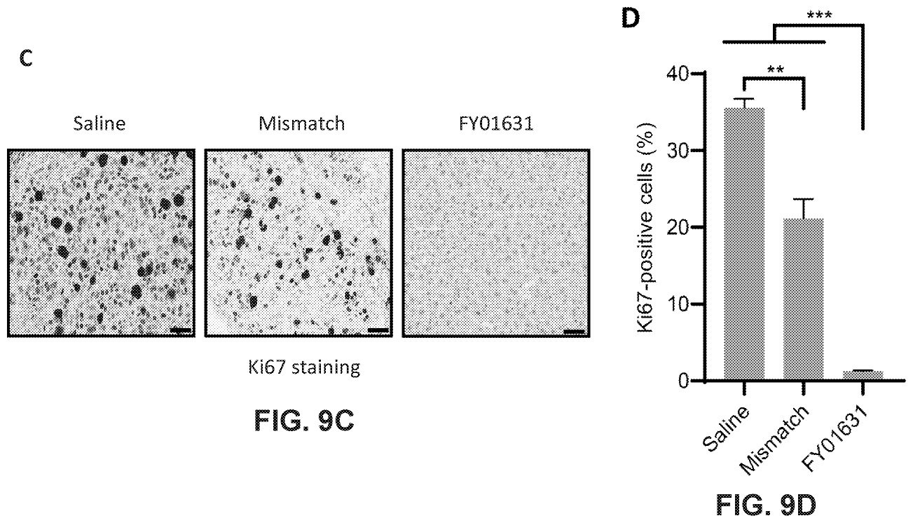

FIG. 9A shows H&E staining images of liver tumor sections. The neoplastic lesions such as pleomorphism and hyperchromatism observed in samples of control groups were improved in FY01631-treated mouse samples. FIG. 9B shows immunohistochemistry of liver tumor sections for EZH2 and H3K27me3 using a specific monoclonal antibody (Abclonal, Wuhan, China) for each protein. FIG. 9C shows immunohistochemistry of liver tumor sections for Ki67 with quantitation on the right (FIG. 9D); **P<0.01, ***P<0.001 (n=4). Tissue samples were collected at day 20 as indicated in FIG. 8A. As shown in FIGS. 9B-9C, levels of EZH2, H3K27me3, and Ki67 in liver tumor samples were all lower in FY01631-treated mice compared to the saline and Mismatch controls. Decreased Ki67 staining indicates that FY01631 markedly inhibited the proliferation of tumor cells. Nuclei were counterstained by hematoxylin.

Those having ordinary skill in the art will appreciate that the disclosure herein can be modified in ways not specifically described herein. The disclosure herein is not to be limited in scope by the specific embodiments described herein, which are for illustrative purposes only. The disclosure includes any modifications and variations, including all functionally equivalent productions, compositions, and methods.

The entire disclosures of all publications cited herein are hereby incorporated by reference. No admission is made that any such publication constitutes prior art or is part of the common general knowledge of those having ordinary skill in the art.

### Example 6—Ameliorated Kidney Diseases in Mouse Models

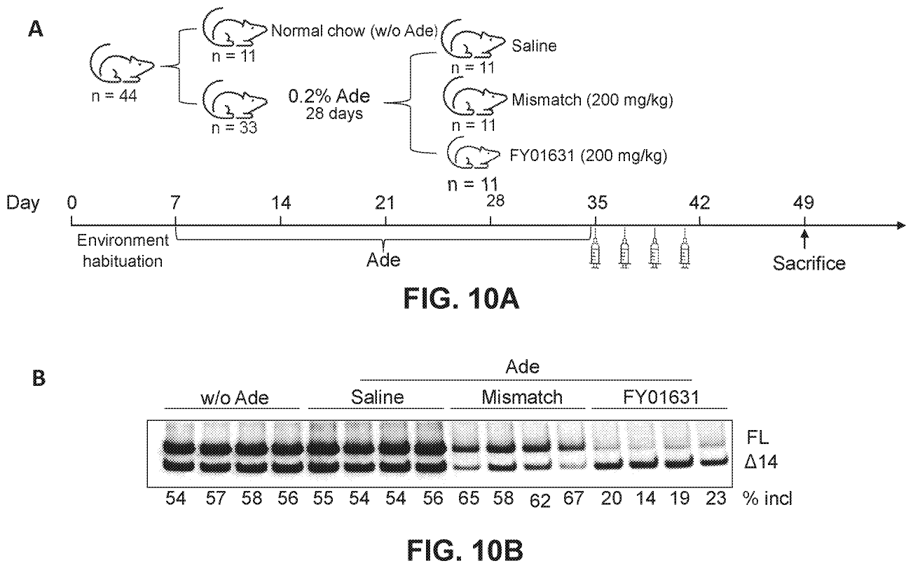

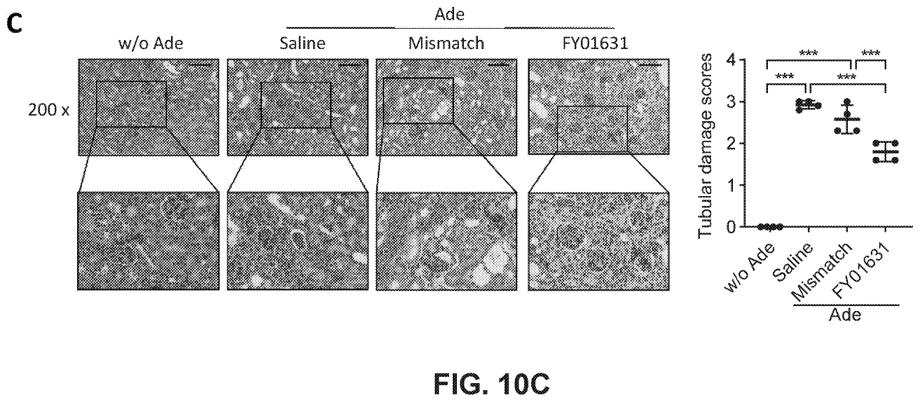

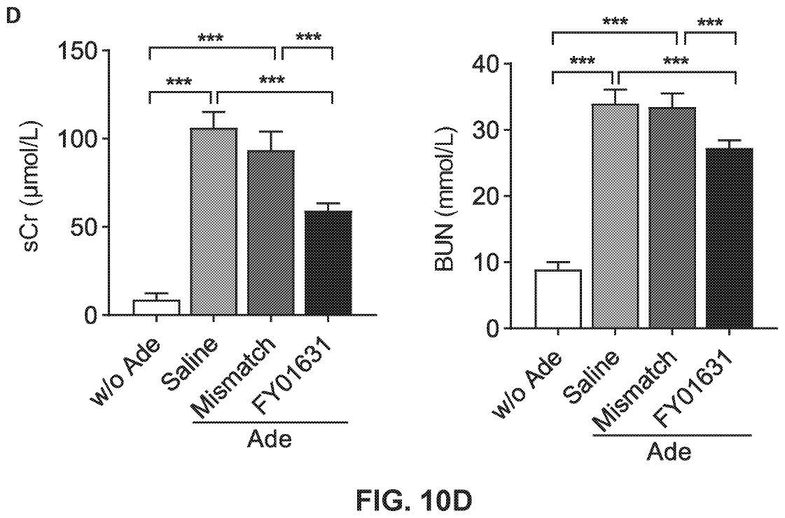

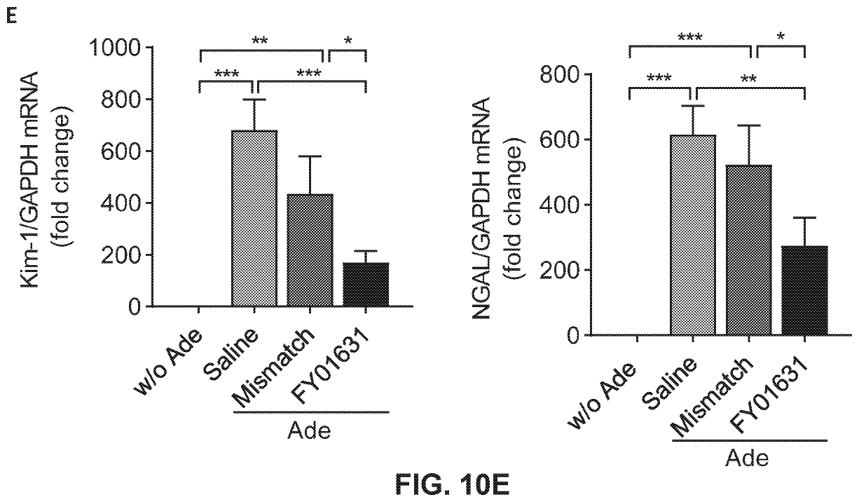

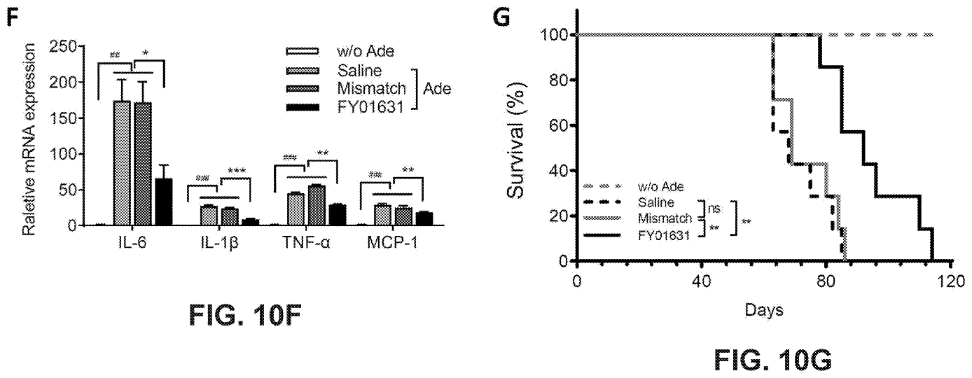

Thirty-three mice were treated with adenine (Ade, 0.2%, 28 days) induce chronic kidney disease (CKD) in mice (the CKD mice). The CKD mice were treated with saline as a negative control (n=11), Mismatch (200 mg/kg, n=11), or FY01631 (200 mg/kg, n=11) once every day for four days (on days 35, 37, 39, and 41; 4 injections total), and sacrificed 8 days after the last injection for analysis (FIG. 10A, the experiment design). FIG. 10B shows splicing changes of the mouse Ezh2 gene in kidney samples of FY01631-treated CKD mice (Ade) compared to normal mice (w/o Ade) and CKD mice (Ade) treated with saline and Mismatch, respectively. FIG. 10C displays kidney tissue pathology in CDK mice (Ade) treated with saline and Mismatch, respectively compared to normal mice (w/o Ade) using H&E staining (left panel, the top images in 200×; and the bottom images show enlarged portions of the corresponding top image defined in a black box). FY01631 treatment ameliorated renal pathological features including renal tubular dilation, renal tubular epithelial cell necrosis and detachment (FIG. 10C, left panel). Tubular damage score was semi-quantitatively calculated based on tubular dilation, brush border loss, cast formation, and tubular epithelial cell necrosis and detachment. Tubular damage scores were lower in FY01631-treated CKD mice than those in saline- and Mismatch-treated CKD mice (FIG. 10C, right panel), although all CDK mice (Ade) showed higher tubular damage scores than the normal mice (w/o Ade). FY01631 treatment markedly reduced serum creatinine (sCr) (FIG. 10D, left) and blood urea nitrogen (BUN) (FIG. 10D, right) levels as well as mRNA expression of Kim-1 (FIG. 10E, left), NGAL (FIG. 10E, right), IL-6, IL-1β, TNF-α, and MCP-1 (FIG. 10F) in kidney samples of CKD mice (Ade), suggesting amelioration of renal function and kidney inflammation. More importantly, the lifespan of CKD mice showed statistically significant increase for FY01631 treated CKD mice (FY01631, dark solid line) over CKD mice treated with saline (Saline, dark dash line) or Mismatch (Mismatch, light grey solid line); CKD mice treated with saline (Saline, dark dash line) and Mismatch (Mismatch, light grey solid line) had no significant (ns) difference in lifespan; and the lifespan of the normal mice (w/o ade) is show as light grey dash line (FIG. 10G).

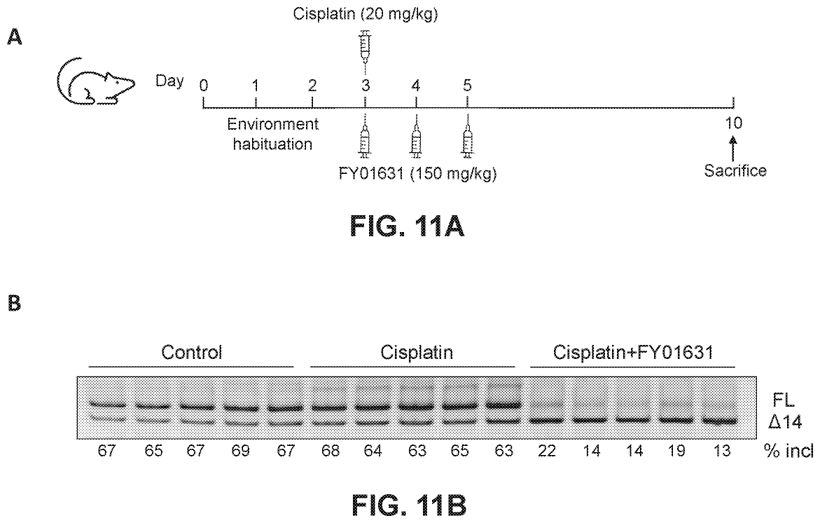

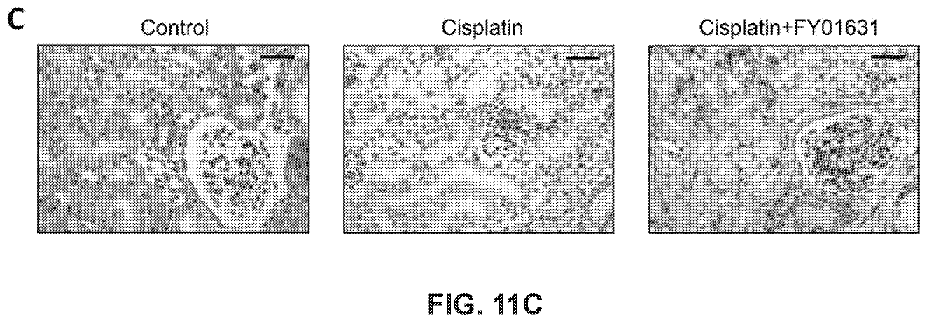

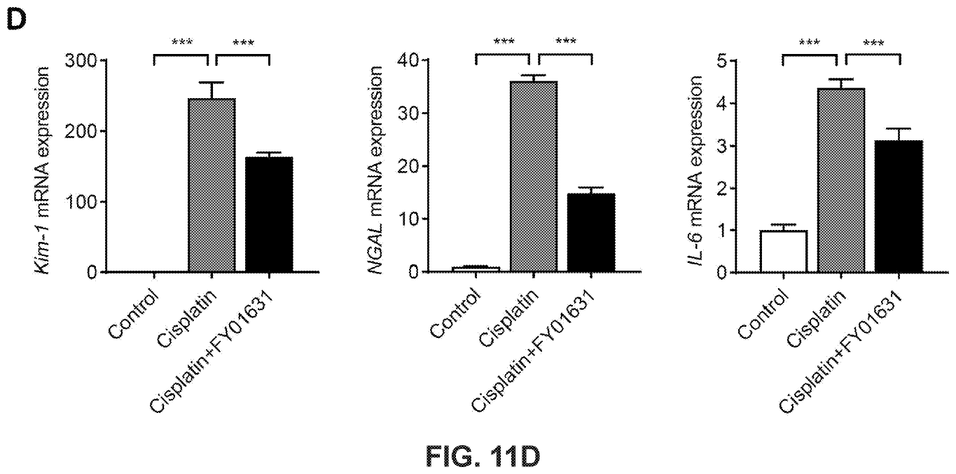

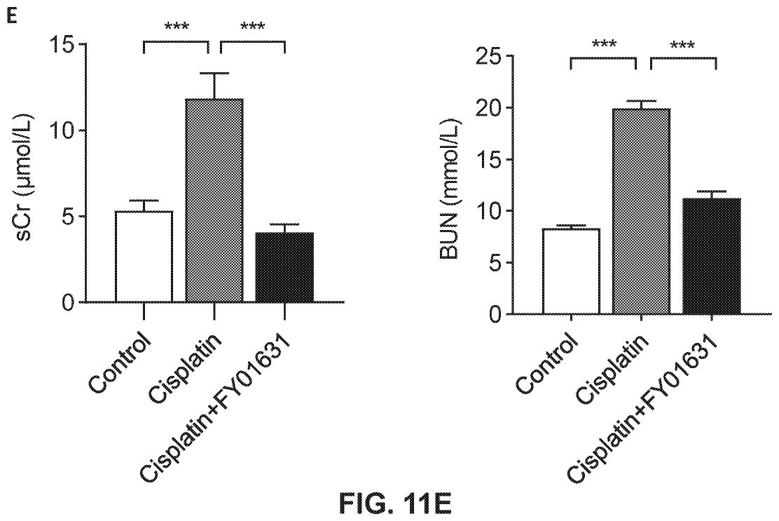

Acute kidney injury (AKI) mouse model was also generated by administration of 20 mg/kg cisplatin dissolved in saline. The AKI mice were then treated with or without FY01631 at 150 mg/kg per day for three consecutive days starting from the same day of the cisplatin treatment, and sacrificed five days after the last FY01631 treatment (FIG. 11A, the experiment design). FIG. 11B shows splicing changes of mouse Ezh2 in normal mice (Control), AKI mice without treatment (Cisplatin), and AKI mice treated with FY01631 (Cisplatin+FY01631). FIG. 11C shows H&E staining of the kidney tissues of normal mice (Control), AKI mice without treatment (Cisplatin), and AKI mice treated with FY01631 (Cisplatin+FY01631), which revealed that FY01631 treatment ameliorated tissue damages in AKI mice. Analysis of mRNA levels of Kim-1, NGAL, and IL-6 in kidney (FIG. 11D) as well as serum creatinine (sCr) and blood urea nitrogen (BUN) (FIG. 11E) indicates that FY01631 improved renal function and alleviated kidney inflammation with statistically significance over AKI mice without treatment (Cisplatin).

1. An antisense oligonucleotide of about 10 to about 30 nucleotides in length comprising all or a portion of a sequence selected from the group consisting of (from 5′ to 3′):
SEQ ID NO: 1
CAAGGCTGCCGTGGATGATCACAG;
SEQ ID NO: 2
CTGCCGTGGATGATCACAGG;
SEQ ID NO: 3
GCTGCCGTGGATGATCACAG;
SEQ ID NO: 4
GGCTGCCGTGGATGATCACA;
SEQ ID NO: 5
AGGCTGCCGTGGATGATCAC;
SEQ ID NO: 6
AAGGCTGCCGTGGATGATCA;
SEQ ID NO: 7
CAAGGCTGCCGTGGATGATC;
SEQ ID NO: 8
ACAAGGCTGCCGTGGATGAT;
SEQ ID NO: 9
CACAAGGCTGCCGTGGATGA;
SEQ ID NO: 10
TCACAAGGCTGCCGTGGATG;
SEQ ID NO: 11
GTCACAAGGCTGCCGTGGAT;
SEQ ID NO: 12
TGTCACAAGGCTGCCGTGGA;
SEQ ID NO: 13
TGCTATCACACAAGGGCACG;
SEQ ID NO: 14
TGTGCTATCACACAAGGGCA;
SEQ ID NO: 15
TTGTGCTATCACACAAGGGC;
SEQ ID NO: 16
TTTGTGCTATCACACAAGGG;
SEQ ID NO: 17
ATTTTGTGCTATCACACAAG;
SEQ ID NO: 18
AATTTTGTGCTATCACACAA;
SEQ ID NO: 20
TTAGAGGAGCCGTCTGAGTA;
SEQ ID NO: 21
TAGTTGTAAACATGGTTAGA;
SEQ ID NO: 23
TGATCACAGGGTTGATAGTT;
SEQ ID NO: 24
GGGCACGAACTGTCACAAGG;
SEQ ID NO: 32
TTTTGTGCTATCACACAAGG;
SEQ ID NO: 33
GCCGTGGATGATCACAGGGTT;
SEQ ID NO: 34
TGCCGTGGATGATCACAGGGT;
SEQ ID NO: 35
CTGCCGTGGATGATCACAGGG;
SEQ ID NO: 36
GCTGCCGTGGATGATCACAGG;
SEQ ID NO: 37
GGCTGCCGTGGATGATCACAG;
SEQ ID NO: 38
AGGCTGCCGTGGATGATCACA;
SEQ ID NO: 39
AAGGCTGCCGTGGATGATCAC;
SEQ ID NO: 40
CAAGGCTGCCGTGGATGATCA;
SEQ ID NO: 41
TGCCGTGGATGATCACAGGGTT;
SEQ ID NO: 42
CTGCCGTGGATGATCACAGGGT;
SEQ ID NO: 43
GCTGCCGTGGATGATCACAGGG;
SEQ ID NO: 44
GGCTGCCGTGGATGATCACAGG;
SEQ ID NO: 45
AGGCTGCCGTGGATGATCACAG;
SEQ ID NO: 46
AAGGCTGCCGTGGATGATCACA;
SEQ ID NO: 47
CAAGGCTGCCGTGGATGATCAC;
SEQ ID NO: 49
GCTGCCGTGGATGATCACAGGGT;
SEQ ID NO: 50
GGCTGCCGTGGATGATCACAGGG;
SEQ ID NO: 51
AGGCTGCCGTGGATGATCACAGG;
SEQ ID NO: 52
AAGGCTGCCGTGGATGATCACAG;
SEQ ID NO: 53
CAAGGCTGCCGTGGATGATCACA;
SEQ ID NO: 54
GCTGCCGTGGATGATCACAGGGTT;
SEQ ID NO: 55
GGCTGCCGTGGATGATCACAGGGT;
SEQ ID NO: 56
AGGCTGCCGTGGATGATCACAGGG;
SEQ ID NO: 57
AAGGCTGCCGTGGATGATCACAGG;
SEQ ID NO: 58
GGCTGCCGTGGATGATCACAGGGTT;
SEQ ID NO: 59
AGGCTGCCGTGGATGATCACAGGGT;
SEQ ID NO: 60
AAGGCTGCCGTGGATGATCACAGGG;
SEQ ID NO: 61
CAAGGCTGCCGTGGATGATCACAGG;
SEQ ID NO: 62
AGGCTGCCGTGGATGATCACAGGGTT;
SEQ ID NO: 63
AAGGCTGCCGTGGATGATCACAGGGT;
SEQ ID NO: 64
CAAGGCTGCCGTGGATGATCACAGGG;
SEQ ID NO: 65
AAGGCTGCCGTGGATGATCACAGGGTT;
SEQ ID NO: 66
CAAGGCTGCCGTGGATGATCACAGGGT;
or
SEQ ID NO: 67
CAAGGCTGCCGTGGATGATCACAGGGTT.

2. The antisense oligonucleotide of claim 1, wherein the nucleotide T can be replaced by nucleotide U.

3. The antisense oligonucleotide of claim 1, wherein the antisense oligonucleotide comprises a non-natural backbone.

4. The antisense oligonucleotide of claim 3, wherein the non-natural backbone comprises modified sugar moieties.

5. The antisense oligonucleotide of claim 4, wherein the modified sugar moieties comprise 2′-O-methoxyethyl ribose moieties (2′-MOE), 2′-O-methyl ribose moieties (2′-OMe), 2′-fluoro ribose moieties (2′-F), 4′-thioribosyl ribose moieties, locked nucleic acid oligos (LNA), constrained ethyl oligos (cEt), and/or morpholino rings.

6. The antisense oligonucleotide of claim 3, wherein the non-natural backbone comprises modified phosphates.

7. The antisense oligonucleotide of claim 6, wherein the modified phosphates comprise phosphorothioates and/or phosphorodiamidates.

8. The antisense oligonucleotide of claim 1, wherein the antisense oligonucleotide comprises modified nitrogenous bases.

9. The antisense oligonucleotide of claim 8, wherein the modified nitrogenous bases comprise 5-methylcytosine bases.

10. A composition comprising: the antisense oligonucleotide of claim 1, and a pharmaceutically acceptable carrier or excipient.

11. A method of promoting exon 14 skipping in the EZH2 gene, inhibiting expression and function of the EZH2 protein, and/or downregulating H3K27me3 expression in vivo, comprising introducing a nucleic acid molecule into a cell, wherein the nucleic acid molecule is the antisense oligonucleotide of claim 1, wherein the antisense oligonucleotide hybridizes at least a portion of CXC (cysteine-rich)-coding region of the EZH2 gene, and wherein the antisense oligonucleotide promotes exon 14 skipping of the EZH2 gene.

12. The method of claim 11, wherein the cell is an animal cell.

13. The method of claim 11, wherein the cell is a human cell.

14. The method of claim 11, wherein the nucleic acid molecule is introduced into a cell by way of an expression vector.

15. The method of claim 14, wherein the expression vector is e.g. an AAV expression vector.

16. A method of treating a condition in a subject comprising administering a therapeutically effective amount of the antisense oligonucleotide of claim 1, wherein the condition is cancer, chronic kidney disease, acute kidney injury, neurodegeneration in ataxia-telangiectasia, retinal degeneration, or inflammation.

17. The method of claim 11, wherein the antisense oligonucleotide is administered via parenteral administration.

18. The method of claim 16, wherein the antisense oligonucleotide is administered via parenteral administration.

19. The method of claim 17, wherein the cancer includes liver cancer, lung cancer, breast cancer, ovarian cancer, endometrial cancer, melanoma, NK/T-cell lymphoma, non-Hodgkin's lymphoma, prostate cancer, bladder cancer, gastric cancer, renal cancer, brain cancer, colorectal cancer, and pancreatic cancer.

20. The method of claim 18, wherein the cancer includes liver cancer, lung cancer, breast cancer, ovarian cancer, endometrial cancer, melanoma, NK/T-cell lymphoma, non-Hodgkin's lymphoma, prostate cancer, bladder cancer, gastric cancer, renal cancer, brain cancer, colorectal cancer, and pancreatic cancer.


SEQUENCE LISTING (67 sequences)

SEQ ID NO: 1 - DNA, 24 bp, synthetic construct
  source 1..24: mol_type = other DNA
caaggctgccgtggatgatcacag

SEQ ID NO: 2 - DNA, 24 bp, synthetic construct
  source 1..24: mol_type = other DNA
gctgccgtggatgatcacagggtt

SEQ ID NO: 3 - DNA, 28 bp, synthetic construct
  source 1..28: mol_type = other DNA
caaggctgccgtggatgatcacagggtt

SEQ ID NO: 4 - DNA, 20 bp, synthetic construct
  source 1..20: mol_type = other DNA
ctgccgtggatgatcacagg

SEQ ID NO: 5 - DNA, 20 bp, synthetic construct
  source 1..20: mol_type = other DNA
gctgccgtggatgatcacag

SEQ ID NO: 6 - DNA, 20 bp, synthetic construct
  source 1..20: mol_type = other DNA
ggctgccgtggatgatcaca

SEQ ID NO: 7 - DNA, 20 bp, synthetic construct
  source 1..20: mol_type = other DNA
aggctgccgtggatgatcac

SEQ ID NO: 8 - DNA, 20 bp, synthetic construct
  source 1..20: mol_type = other DNA
aaggctgccgtggatgatca

SEQ ID NO: 9 - DNA, 20 bp, synthetic construct
  source 1..20: mol_type = other DNA
caaggctgccgtggatgatc

SEQ ID NO: 10 - DNA, 20 bp, synthetic construct
  source 1..20: mol_type = other DNA
acaaggctgccgtggatgat

SEQ ID NO: 11 - DNA, 20 bp, synthetic construct
  source 1..20: mol_type = other DNA
cacaaggctgccgtggatga

SEQ ID NO: 12 - DNA, 20 bp, synthetic construct
  source 1..20: mol_type = other DNA
tcacaaggctgccgtggatg

SEQ ID NO: 13 - DNA, 20 bp, synthetic construct
  source 1..20: mol_type = other DNA
gtcacaaggctgccgtggat

SEQ ID NO: 14 - DNA, 20 bp, synthetic construct
  source 1..20: mol_type = other DNA
tgtcacaaggctgccgtgga

SEQ ID NO: 15 - DNA, 20 bp, synthetic construct
  source 1..20: mol_type = other DNA
tgctatcacacaagggcacg

SEQ ID NO: 16 - DNA, 20 bp, synthetic construct
  source 1..20: mol_type = other DNA
tgtgctatcacacaagggca

SEQ ID NO: 17 - DNA, 20 bp, synthetic construct
  source 1..20: mol_type = other DNA
ttgtgctatcacacaagggc

SEQ ID NO: 18 - DNA, 20 bp, synthetic construct
  source 1..20: mol_type = other DNA
tttgtgctatcacacaaggg

SEQ ID NO: 19 - DNA, 20 bp, synthetic construct
  source 1..20: mol_type = other DNA
attttgtgctatcacacaag

SEQ ID NO: 20 - DNA, 20 bp, synthetic construct
  source 1..20: mol_type = other DNA
aattttgtgctatcacacaa

SEQ ID NO: 21 - DNA, 24 bp, synthetic construct
  source 1..24: mol_type = other DNA
catgggtgtcgcgggtggtctcac

SEQ ID NO: 22 - DNA, 176 bp, synthetic construct
  source 1..176: mol_type = other DNA
cctgcatgatgttatctttactcagacggctcctctaaccatgtttacaactatcaaccctgtgatcatccacggcagccttgtgacagttcgtgcccttgtgtgatagcacaaaatttttgtgaaaagttttgtcaatgtagttcagagtgtaagtatttgttgctttgatgcaa

SEQ ID NO: 23 - DNA, 20 bp, synthetic construct
  source 1..20: mol_type = other DNA
ttagaggagccgtctgagta

SEQ ID NO: 24 - DNA, 20 bp, synthetic construct
  source 1..20: mol_type = other DNA
tagttgtaaacatggttaga

SEQ ID NO: 25 - DNA, 20 bp, synthetic construct
  source 1..20: mol_type = other DNA
tgatcacagggttgatagtt

SEQ ID NO: 26 - DNA, 20 bp, synthetic construct
  source 1..20: mol_type = other DNA
gggcacgaactgtcacaagg

SEQ ID NO: 27 - DNA, 20 bp, synthetic construct
  source 1..20: mol_type = other DNA
ttttcacaaaaattttgtgc

SEQ ID NO: 28 - DNA, 20 bp, synthetic construct
  source 1..20: mol_type = other DNA
ctacattgacaaaacttttc

SEQ ID NO: 29 - DNA, 20 bp, synthetic construct
  source 1..20: mol_type = other DNA
tacttacactctgaactaca

SEQ ID NO: 30 - DNA, 20 bp, synthetic construct
  source 1..20: mol_type = other DNA
tatcacacaagggcacgaac

SEQ ID NO: 31 - DNA, 20 bp, synthetic construct
  source 1..20: mol_type = other DNA
ctatcacacaagggcacgaa

SEQ ID NO: 32 - DNA, 20 bp, synthetic construct
  source 1..20: mol_type = other DNA
gctatcacacaagggcacga

SEQ ID NO: 33 - DNA, 20 bp, synthetic construct
  source 1..20: mol_type = other DNA
gtgctatcacacaagggcac

SEQ ID NO: 34 - DNA, 20 bp, synthetic construct
  source 1..20: mol_type = other DNA
ttttgtgctatcacacaagg

SEQ ID NO: 35 - DNA, 21 bp, synthetic construct
  source 1..21: mol_type = other DNA
ctgccgtggatgatcacaggg

SEQ ID NO: 36 - DNA, 21 bp, synthetic construct
  source 1..21: mol_type = other DNA
gctgccgtggatgatcacagg

SEQ ID NO: 37 - DNA, 21 bp, synthetic construct
  source 1..21: mol_type = other DNA
ggctgccgtggatgatcacag

SEQ ID NO: 38 - DNA, 21 bp, synthetic construct
  source 1..21: mol_type = other DNA
aggctgccgtggatgatcaca

SEQ ID NO: 39 - DNA, 21 bp, synthetic construct
  source 1..21: mol_type = other DNA
aaggctgccgtggatgatcac

SEQ ID NO: 40 - DNA, 21 bp, synthetic construct
  source 1..21: mol_type = other DNA
caaggctgccgtggatgatca

SEQ ID NO: 41 - DNA, 22 bp, synthetic construct
  source 1..22: mol_type = other DNA
tgccgtggatgatcacagggtt

SEQ ID NO: 42 - DNA, 22 bp, synthetic construct
  source 1..22: mol_type = other DNA
ctgccgtggatgatcacagggt

SEQ ID NO: 43 - DNA, 22 bp, synthetic construct
  source 1..22: mol_type = other DNA
gctgccgtggatgatcacaggg

SEQ ID NO: 44 - DNA, 22 bp, synthetic construct
  source 1..22: mol_type = other DNA
ggctgccgtggatgatcacagg

SEQ ID NO: 45 - DNA, 22 bp, synthetic construct
  source 1..22: mol_type = other DNA
aggctgccgtggatgatcacag

SEQ ID NO: 46 - DNA, 22 bp, synthetic construct
  source 1..22: mol_type = other DNA
aaggctgccgtggatgatcaca

SEQ ID NO: 47 - DNA, 22 bp, synthetic construct
  source 1..22: mol_type = other DNA
caaggctgccgtggatgatcac

SEQ ID NO: 48 - DNA, 23 bp, synthetic construct
  source 1..23: mol_type = other DNA
ctgccgtggatgatcacagggtt

SEQ ID NO: 49 - DNA, 23 bp, synthetic construct
  source 1..23: mol_type = other DNA
gctgccgtggatgatcacagggt

SEQ ID NO: 50 - DNA, 23 bp, synthetic construct
  source 1..23: mol_type = other DNA
ggctgccgtggatgatcacaggg

SEQ ID NO: 51 - DNA, 23 bp, synthetic construct
  source 1..23: mol_type = other DNA
aggctgccgtggatgatcacagg

SEQ ID NO: 52 - DNA, 23 bp, synthetic construct
  source 1..23: mol_type = other DNA
aaggctgccgtggatgatcacag

SEQ ID NO: 53 - DNA, 23 bp, synthetic construct
  source 1..23: mol_type = other DNA
caaggctgccgtggatgatcaca

SEQ ID NO: 54 - DNA, 24 bp, synthetic construct
  source 1..24: mol_type = other DNA
gctgccgtggatgatcacagggtt

SEQ ID NO: 55 - DNA, 24 bp, synthetic construct
  source 1..24: mol_type = other DNA
ggctgccgtggatgatcacagggt

SEQ ID NO: 56 - DNA, 24 bp, synthetic construct
  source 1..24: mol_type = other DNA
aggctgccgtggatgatcacaggg

SEQ ID NO: 57 - DNA, 24 bp, synthetic construct
  source 1..24: mol_type = other DNA
aaggctgccgtggatgatcacagg

SEQ ID NO: 58 - DNA, 25 bp, synthetic construct
  source 1..25: mol_type = other DNA
ggctgccgtggatgatcacagggtt

SEQ ID NO: 59 - DNA, 25 bp, synthetic construct
  source 1..25: mol_type = other DNA
aggctgccgtggatgatcacagggt

SEQ ID NO: 60 - DNA, 25 bp, synthetic construct
  source 1..25: mol_type = other DNA
aaggctgccgtggatgatcacaggg

SEQ ID NO: 61 - DNA, 25 bp, synthetic construct
  source 1..25: mol_type = other DNA
caaggctgccgtggatgatcacagg

SEQ ID NO: 62 - DNA, 26 bp, synthetic construct
  source 1..26: mol_type = other DNA
aggctgccgtggatgatcacagggtt

SEQ ID NO: 63 - DNA, 26 bp, synthetic construct
  source 1..26: mol_type = other DNA
aaggctgccgtggatgatcacagggt

SEQ ID NO: 64 - DNA, 26 bp, synthetic construct
  source 1..26: mol_type = other DNA
caaggctgccgtggatgatcacaggg

SEQ ID NO: 65 - DNA, 27 bp, synthetic construct
  source 1..27: mol_type = other DNA
aaggctgccgtggatgatcacagggtt

SEQ ID NO: 66 - DNA, 27 bp, synthetic construct
  source 1..27: mol_type = other DNA
caaggctgccgtggatgatcacagggt

SEQ ID NO: 67 - DNA, 28 bp, synthetic construct
  source 1..28: mol_type = other DNA
caaggctgccgtggatgatcacagggtt